(19) United States
(12) Patent Application Publication
(10) Pub. No.: US 2003/0086932 A1
(43) Pub. Date: May 8, 2003

(54) Surface-bound antigen binding portions of antibodies that bind to CTLA-4 and CD28 and uses therefor

(76) Inventors: Jeffrey A. Bluestone, San Francisco, CA (US); Mary Collins, Natick, MA (US); Matthew J. Whitters, Hudson, MA (US); Matthew D. Griffin, Rochester, MN (US); David M. Kranz, Champaign, IL (US)

Correspondence Address: LAHIVE & COCKFIELD, 28 STATE STREET, BOSTON, MA, 02109, US

(21) Appl. No.: 09/835,297

(22) Filed: Apr. 12, 2001

Related U.S. Application Data
(60) Provisional application No. 60/196,851, filed on Apr. 12, 2000.

(51) Int. Cl. A61K 39/395; C07K 16/46

(52) U.S. Cl. 424/178.1; 530/391.1

(57) ABSTRACT

The instant invention provides compositions for downmodulation or upmodulation of the immune response. For example, constructs comprising an exposed surface having attached to it i) an antigen-binding portion of an antibody that binds to a CTLA-4 or CD28 molecule that is expressed on a T cell of the subject, and ii) an MHC molecule selected from the group consisting of: a class II molecule that is syngeneic to the subject, a class I molecule that is syngeneic to the subject, and a class I molecule that is allogeneic to the subject are provided. Methods of using the molecules of the invention in downmodulation or upmodulation of the immune response are also provided. Also provided are methods of downmodulating a immune response in a subject comprising causing a cell of the subject to express an antigen-binding portion of an antibody that binds a CTLA-4 or CD28 molecule that is expressed on a T cell of the subject, such that the immune response in the subject is dowmnodulated. Methods of preparing an allogeinc cell for transplantation and methods of transplanting engineered allogeinc cells are also provided.

### BACKGROUND OF THE INVENTION

In order for T cells to respond to foreign proteins, two signals must be provided by antigen-presenting cells (APCs) to resting T lymphocytes (Jenkins, M. and Schwartz, R. (1987) J. Exp. Med. 165, 302-319; Mueller, D. L., et al. (1990) J. Immunol. 144, 3701-3709). The first signal, which confers specificity to the immune response, is transduced via the T cell receptor (TCR) following recognition of foreign antigenic peptide presented in the context of the major histocompatibility complex (MHC). Polyclonal activators (e.g., anti-CD3 antibodies can also be used to transmit primary activation signals. The second signal, termed costimulation, induces T cells to proliferate and become functional (Lenschow et al. 1996. Annu. Rev. Immunol. 14:233). Costimulation is neither antigen-specific, nor MHC restricted and is thought to be provided by one or more distinct cell surface molecules expressed by APCs (Jenkins, M. K., et al. 1988 J. Immunol. 140, 3324-3330; Linsley, P. S., et al. 1991 J. Exp. Med 173, 721-730; Gimmi, C. D., et al., 1991 Proc. Natl. Acad. Sci. USA. 88, 6575-6579; Young, J. W., et al. 1992 J. Clin. Invest. 90, 229-237; Koulova, L., et al. 1991 J. Exp. Med. 173, 759-762; Reiser, H., et al. 1992 Proc. Natl. Acad. Sci. USA. 89, 271-275; van-Seventer, G. A., et al. (1990) J. Immunol. 144, 4579-4586; LaSalle, J. M., et al., 1991 J. Immunol. 147, 774-80; Dustin, M. I., et al., 1989 J. Exp. Med. 169, 503; Armitage, R. J., et al. 1992 Nature 357, 80-82; Liu, Y., et al. 1992 J. Exp. Med. 175, 437-445).

The CD80 (B7-1) and CD86 (B7-2) proteins, expressed on APCs, are critical costimulatory molecules (Freeman et al. 1991. J. Exp. Med. 174:625; Freeman et al. 1989 J. Immunol. 143:2714; Azuma et al. 1993 Nature 366:76; Freeman et al. 1993. Science 262:909). B7-2 appears to play a predominant role during primary immune responses, while B7-1, which is upregulated later in the course of an immune response, may be important in prolonging primary T cell responses or costimulating secondary T cell responses (Bluestone. 1995. Immunity. 2:555).

One ligand to which B7-1 and B7-2 bind, CD28, is constitutively expressed on resting T cells and increases in expression after activation. After signaling through the T cell receptor, ligation of CD28 and transduction of a second, costimulatory signal induces T cells to proliferate and secrete IL-2 (Linsley, P. S., et al. 1991 J. Exp. Med. 173, 721-730; Gimmi, C. D., et al. 1991 Proc. Natl. Acad. Sci. USA. 88, 6575-6579; June, C. H., et al. 1990 Immunol. Today. 11, 211-6; Harding, F. A., et al. 1992 Nature. 356, 607-609).

The two signal model of T cell activation has led to the development of new therapeutic approaches in the treatment of immunological disorders. Instead of globally inhibiting T and B cell function with reagents such as steroids, calcineurin inhibitors, and pan-reactive monoclonal antibodies, investigators are now targeting a variety of costimulatory pathways in the hope of developing a more antigen specific immunotherapy. Recent studies have shown that T cell activation can be augmented, and in some instances antigen-specific immunization can be induced, when the CD28 co-stimulatory pathways are engaged with B7 reagents (e.g., B7 expressed on the surface of tumor cells). The engagement of CD28 by either B7-1 or B7-2 at the time of TCR engagement with antigen/MHC results in T cell activation and lymphokine production. In contrast, TCR engagement in the absence of CD28 ligation promotes apoptosis, reduces cell expansion, and can induce a state of antigen-specific non-responsiveness, termed anergy. Thus, the selective activation of the CD28 pathway in an antigen-specific manner can lead to potent immunity.

A second ligand, termed CTLA-4 (CD 152) is homologous to CD28 but is not expressed on resting T cells and appears following T cell activation (Brunet, J. F., et al., 1987 Nature 328, 267-270). CTLA-4 appears to be critical in negative regulation of T cell responses (Waterhouse et al. 1995. Science 270:985). Blockade of CTLA-4 has been found to remove inhibitory signals, while aggregation of CTLA-4 has been found to provide inhibitory signals that downregulate T cell responses (Allison and Krummel. 1995. Science 270:932). The B7 molecules have a higher affinity for CTLA-4 than for CD28 (Linsley, P. S., et al., 1991 J. Exp. Med. 174, 561-569) and B7-1 and B7-2 have been found to bind to distinct regions of the CTLA-4 molecule and have different kinetics of binding to CTLA-4 (Linsley et al. 1994. Immunity. 1:793).

CTLA-4/B7 is now recognized as imposing a negative effect on cell cycle progression, IL-2 production, and proliferation of T cells following activation. Mice lacking CTLA-4 through targeted gene disruption demonstrate a remarkable dysregulation of T cell homeostasis and die within four weeks of birth from unchecked lymphoproliferative disease. Characterization of molecular mechanisms underlying negative immune regulation by CTLA-4 in murine systems have revealed their essential role in the maintenance of immune homeostasis, the prevention of autoimmunity, and the orchestration of effective cellular and humoral responses. This critical role has been borne out by studies in which CTLA-4/B7 interactions have been blocked in vivo through administration of monoclonal antibody. A role for CTLA-4 in the intensity of all responses to transplanted organs has also been suggested by blocking studies. In addition, murine studies of antigen-specific T cell tolerance have shown that CTLA-4 function is necessary for unresponsiveness to subsequent antigen exposure. Furthermore, evidence continues to accumulate that these same mechanisms operate in the human and are linked to the pathogenesis of immune-mediated disease.

Thus, an intimate and intricate relationship that exists between positive and negative regulation during the generation of immune responses is exemplified by the functional profiles of the homologous proteins CD28 and CTLA-4 (CD152). Despite their structural similarities and shared affinity for the ligands B7-1 (CD80) and B7-2 (CD86), it is now clear that the two proteins mediate essentially opposing effects on T cell activation, whereas the CD28/B7 interaction is known to serve as a positive co-stimulator in the context of TCR engagement by MHC/antigen complex. That CTLA-4 mediated negative regulation can critically affect the course of disease processes such as neoplasia, infection, and autoimmunity. While this implies that enhancement of CTLA-4 function might be used to induce immune hyporesponsiveness or tolerance to disease related antigens, the pursuit of such a strategy is complicated by the shared affinity of CTLA-4 and CD28 for their natural ligands as well as by incomplete understanding of the mechanisms underlying the negative regulatory pathway.

Within the past decade there has been a growing appreciation of the problems associated with regulating these co-stimulatory pathways as the same ligands (B7-1 and B7-2) are able to interact with both the positive and negative regulatory receptors, CD28 and CTLA-4, respectively. Examples of the potency and complexity of these pathways has been exemplified in multiple in vivo model systems. Blockade of the CD28 costimulatory pathway results in the development of antigen specific tolerance in murine and human systems (Harding, F. A., et al. 1992 Nature. 356, 607-609; Lenschow, D. J., et al., 1992 Science. 257, 789-792; Turka, L. A., et al. (1992) Proc. Natl. Acad. Sci. USA. 89, 11102-11105; Gimmi, C. D., et al. (1993) Proc. Natl. Acad. Sci USA 90, 6586-6590; Boussiotis, V., et al. (1993) J. Exp. Med. 178, 1753-1763). Conversely, expression of B7 by B7 negative murine tumor cells induces T-cell mediated specific immunity accompanied by tumor rejection and long lasting protection to tumor challenge (Chen, L., et al. (1992) Cell 71, 1093-1102; Townsend, S. E. and Allison, J. P. (1993) Science 259, 368-370; Baskar, S., et al. (1993) Proc. Natl. Acad. Sci. 90, 5687-5690).

However, the results can be quite different in certain immune settings. Under some conditions, treatment of autoimmune-prone mice with a B7 antagonist affects CD28/B7 interactions preventing the development of diabetes while in other settings the same B7 antagonist blocks CTLA-4/B7 interactions in the animals leading to the development of severe disease. Owing to the opposing effects of engagement of CD28 and CTLA-4, specific compositions and methods for separately manipulating positive and negative regulation of T cells would be of great benefit. Such specific manipulation of positive and negative signaling pathways in T cells would offer great potential to selectively stimulate or suppress immune responses.

### SUMMARY OF THE INVENTION

CD28 and CTLA-4 have been shown to be important in regulating T cell activation and the induction of immune tolerance. Development of strategies to reliably engage these specific T cell surface marker and, in so doing, specifically promote T cell inhibition has been difficult. This is particularly true in the case of CTLA-4 because it shares its ligands, B7-1 (CD80) and B7-2 (CD86), with CD28. In addition, CTLA-4 is only transiently expressed on activated T cells, thus, attempts to manipulate signaling via the molecule may not result in effective inhibition of T cells. Moreover, soluble antibodies that recognize CTLA-4 in the absence of concomitant T cell receptor co-litagion have been shown to stimulate, rather than reduce, immune responses (U.S. Pat. No. 5,811,097).

The instant invention is based, at least in part, on the development of a surface-linked antigen-binding portions of antibodies (e.g., scFv) which bind to CTLA-4 or CD28. Such antigen-binding portions were shown to be expressed at the cell surface following transfection into cells and to specifically regulate proliferation and IL-2 production of both CD4+ and CD8+T cells. Most critically, these agents regulate these functions in a potent and predictable manner.

Other functional parameters of T cell activation were also shown to be affected by the antigen-binding portion of anti-CTLA-4 antibody. For example, engagement of CTLA-4 with CTLA-4scFv resulted in reduction of tyrosine phosphorylation of components of the proximal TCR signaling apparatus (p23 TCRζ) and Linker for Activated T cells (p36 Lat) during co-incubation of pre-activated T cells with transfectants bearing CTLA-4scFv. The CD28scFv had the opposite effect, synergizing with anti-CD3 mAbs to increase the tyrosine phosphorylation of components of the proximal TCR signaling apparatus (p23 TCRζ) and p36 Lat during co-incubation of pre-activated T cells. The ability of CTLA-4 engagement to negatively regulate primary and secondary activation of antigen-specific CD4+T cells was optimal when co-expressed with MHC/peptide complex on the same cell surface. This occurred in vivo as well; expression of cell surface CD28 was shown to promote anti-tumor responses in vivo.

Accordingly, in one aspect, the invention provides a construct for downmodulating an immune response in a subject, comprising an exposed surface, wherein said exposed surface has attached to it i) an antigen-binding portion of an antibody that binds to a CTLA-4 molecule that is expressed on a T cell of the subject, and ii) an MHC molecule selected from the group consisting of: a class II molecule that is syngeneic to the subject, a class I molecule that is syngeneic to the subject, and a class I molecule that is allogeneic to the subject.

In one embodiment, the antigen-binding portion is a single chain Fv (scFv) molecule. In another embodiment, the single chain Fv (scFv) molecule binds to human CTLA-4. In another embodiment, the scFv molecule is humanized.

In yet another embodiment, the antigen binding cleft of the MHC molecule comprises a peptide for which the immune response is specific.

In one embodiment, the construct comprises a lipid bilayer. In one embodiment, the construct is an acellular construct. In one embodiment, the construct is a cell. In yet another embodiment, the cell is a eukaryotic cell. In on embodiment, the cell is syngeneic to the subject. In one embodiment, the cell is allogeneic to the subject.

In one embodiment, the antigen-binding portion of an antibody that binds to a CTLA-4 molecule is attached to the exposed surface via a phosphatidylinositol-glycan anchor. In another embodiment, the antigen-binding portion of an antibody that binds to a CTLA-4 molecule is attached to the exposed surface via a transmembrane domain. In another embodiment, the antigen-binding portion of an antibody that binds to a CTLA-4 molecule is attached to the exposed surface via a chemical linkage.

In one embodiment, the construct does not bind to CD28.

In one embodiment, the invention pertains to a method of downmodulating a primary immune response in a subject comprising administering a construct to the subject such that an immune response in the subject is downmodulated. In another embodiment, the invention pertains to a method of downmodulating an ongoing immune response in a subject comprising administering the construct of claim 1 to the subject such that an immune response in the subject is downmodulated.

In one aspect, the invention pertains to a method of downmodulating a immune response in a subject comprising causing a cell of the subject to express an antigen-binding portion of an antibody that binds a CTLA-4 molecule, the CTLA-4 molecule that is expressed on a T cell of the subject, such that the immune response in the subject is downmodulated.

In one embodiment, the antigen-binding portion is a single chain Fv (scFv) molecule. In one embodiment, the single chain Fv (scFv) molecule binds to human CTLA-4. In one embodiment, the scFv molecule is humanized.

In one embodiment, the immune response is against an self antigen. In one embodiment, the immune response is against an non-self antigen. In one embodiment, the immune response is against an allogeneic antigen.

In one embodiment, the immune response is mediated by CD4+ T cells. In another embodiment, the immune response is mediated by CD8+ T cells.

In one embodiment, the cell is a professional antigen presenting cell.

In one embodiment, the cell is further caused to express an MHC class I or an MHC class II molecule.

In one embodiment, the cell is transfected with a nucleic acid molecule encoding the antigen-binding portion of an antibody that binds CTLA-4. In one embodiment, the cell is transfected ex vivo. In one embodiment, the cell is transfected in vivo.

In one aspect, the invention pertains to a method of preparing an allogeinc cell for transplantation into a subject comprising causing the allogeneic cell to express an antigen-binding portion of an antibody that binds a CTLA-4 molecule expressed on a T cell of the subject to thereby prepare an allogeneic cell for transplantation into a subject.

In another aspect, the invention pertains to a method of transplanting an engineered allogeinc cell to a subject comprising: causing an allogeneic cell to express an antigen-binding portion of an antibody that binds a CTLA-4 molecule on a T cell of the subject to create an engineered allogeneic cell, and administering the engineered allogeneic cell to the subject such that the engineered allogeneic cell is transplanted to the subject.

### BRIEF DESCRIPTION OF THE DRAWINGS

FIG. 1 shows the strategy for construction of surface-linked scFvs and flow cytometric analysis of 293 cells transfected with CTLA-4 and anti-CD28scFv constructs.

FIG. 2 shows that anti-CTLA-4 scFv attenuates the primary activation of murine T cells by combined anti-CD3, and anti-CD28scFvs.

FIG. 3 shows that anti-CTLA-4scFv reduced proliferation and cytokine production during secondary stimulation of pre-activated murine T cells.

FIG. 4 shows that both CD4+ and CD8+T cells can be negatively regulated by selective CTLA-4 engagement during secondary activation.

FIG. 5 shows that co-ligation of TCR and CTLA-4 by surface-linked scFvs results in attenuated tyrosine phosphorylation of proximal TCR signaling components.

FIG. 6 shows that negative regulation of T cell activation events requires co-expression of anti-CD3εscFv and anti-CTLA-4scFv on the same cell surface.

FIG. 7 shows anti-CTLA-4scFv attenuates antigen-induced activation of resting and pre-activated DO11.10 TCR transgenic CD4+T cells.

### DETAILED DESCRIPTION OF THE INVENTION

As set forth briefly above, the instant invention pertains, at least in part, to constructs comprising a surface-linked antigen-binding portion of an antibody that binds to CTLA-4 or CD28 linked to a T cell receptor binding moiety, and to methods of using such active molecules of the invention to modulate the immune response.

Various aspects of the invention are described in further detail in the following subsections:

I. Definitions

As used herein, the term “construct” includes molecules for modulating the immune response in a subject which comprise a surface that, upon introduction into a subject, would be exposed to the extracellular milieu. As described in more detail below, such constructs can be cellular or acellular in nature.

As used herein, the term “acellular construct’ includes constructs of the invention which are not cellular in nature, e.g., are not prokaryotic or eukaryotic cells.

As used herein, the term “GPI anchor” includes glycosylphosphatidylinositol (phosphotidylinositol-glycan) (GPI) linkages that anchor polypeptides to surfaces. These anchors link polypeptides via an oligosaccharide linkage to the phospholipid, phosphitidylinositol. Such linkages can be cleaved by phosphatidyl-inositol-specific phospholipase C.

As used herein, the term “transmembrane domain” includes a hydrophobic region of a polypeptide that interacts with the hydrophobic tails of lipid molecules in a lipid bilayer. As used herein, the term “lipid bilayer” includes those structures formed by amphipathic lipid molecules in aqueous solution. Lipid bilayers can comprise any of a variety of lipid molecules, e.g., cholesterol, phosphatidyl-ethanolamine, phosphatidylserine, phosphatidylcholine, sphingomyelin, glycolipids, etc. The composition of the membrane can be altered as appropriate to achieve the desired fluidity of the membrane. Lipid bilayers of the invention may comprise glycolipids.

As used herein, the term “T cell” includes CD4+(CD4+ bearing) T cells and CD8+(CD8 bearing) T cells. CD8+(cytotoxic T cells) primarily recognize antigenic peptides in the context of MHC class I molecules. CD4+cells (predominantly helper T cells) primarily recognize antigenic peptides in the context of MHC class II molecules. The term T cell also includes both T helper 1 type T cells and T helper 2 type T cells.

As used herein, the term “T cell receptor binding moiety” includes groups that bind to T cell receptors (e.g., antibody binding portions of antibodies or MHC molecules)

The major histocompatibility complex (MHC) is a cluster of genetic loci that encode three different classes of polypeptide products (class I, II, and III). Class I and II MHC proteins are involved in the presentation of antigens to T cells. Class II molecules are involved in the activation of antigen-specific MHC-restricted T helper cells, which in turn activate cytotoxic T lymphocytes and antibody-producing B cells. The human MHC on chromosome 6 is termed “HLA” (human leukocyte antigen). In humans, there are at least three types of Class II molecules, HLA-DR, DQ, and DP. The HLA region is highly polymorphic and the combination of alleles at HLA loci in any individual determines “self” HLA.

The term “subject” as used herein refers to vertebrate hosts, particularly to mammals, and includes, but is not limited to, primates, including humans, and domestic animals.

As used herein the term “syngeneic” includes cells which have the same HLA specificity as those of a subject. Preferably, syngeneic cells are autologous, i.e., from the same individual in whom an immune response is to be downmodulated.

As used herein the term “allogeneic” includes cells which have a different HLA specificity than the subject, i.e., are from different individuals of the same species, e.g., from humans other than the subject in whom an immune response is to be downmodulated.

The term “antigen presenting cell” includes “professional antigen presenting cells” that constitutively express MHC class II molecules (e.g., B lymphocytes, monocytes, dendritic cells, Langerhans cells, and activated T cells in humans) as well as other antigen presenting cells that are capable of presenting antigen to T cells. APCs can express the appropriate combination of MHC molecules and costimulatory and/or adhesion molecules known in the art to be sufficient for presentation of antigen to T cells or can be induced or engineered to express such molecules.

As used herein, the term “immune response” includes T cell mediated and/or B cell mediated immune responses that are influenced by modulation of T cell costimulation. Exemplary immune responses include T cell responses, e.g., proliferation, cytokine production, and cellular cytotoxicity. In addition, the term immune response includes immune responses that are indirectly effected by T cell activation, e.g., antibody production (humoral responses) and activation of cytokine responsive cells, e.g., macrophages.

As used herein, the term “primary immune response” includes immune responses to antigens which have not been seen before by a subject, e.g., to which the subject is naive.

As used herein, the term “secondary immune response” includes immune responses to antigens which have been seen before by a subject, e.g., to which the subject has been primed. The tem “ongoing immune response” includes an immune response to a certain antigen which is ongoing, e.g., is presently active and detectable.

As used herein, the term “exogenous” with reference to a peptide includes peptides that are extracellular and e.g., are generated in or taken up by endocytic pathways (lysosomal or endosomal pathways) and become associated primarily with class II molecules. As used herein, the term “endogenous” with reference to a peptide includes peptides which enter or are produced by the endoplasmic reticulum of a cell, e.g., which are derived primarily from the cytoplasm of a cell and which are presented primarily in the context of class I molecules.

As used here, the term “self” with reference to a peptide includes peptides which are not foreign to a subject and to which an autoimmune response can occur. The immune system can normally discriminate between self and non-self (“foreign”). Optimally, the mammalian immune system is non-reactive (e.g., tolerant) to self-antigens. The mechanisms that provide tolerance normally eliminate or render inactive clones of B and T cells that would otherwise carry out anti-self reactions. Autoimmune diseases (e.g., multiple sclerosis, rheumatoid arthritis, lupus erythematosus, and Type 2 diabetes mellitus) represent an aberrant immune attack in which antibodies or T cells of a host are directed against self-antigen not normally the target of the immune response. Autoimmunity results from the dysfunction of normal mechanisms of self-tolerance that prevent the production of functional self-reactive clones of B and T cells.

As used herein, the term “costimulate” with reference to activated T cells includes the ability of a costimulatory molecule to provide a second, non-activating receptor mediated signal (a “costimulatory signal”) that induces proliferation or effector function. For example, a costimulatory signal can result in cytokine secretion, e.g., in a T cell that has received a T cell-receptor-mediated signal. T cells that have received a cell-receptor mediated signal, e.g., via a T cell receptor (TCR) (e.g., by an antigen or by a polyclonal activator) are referred to herein as “activated T cells.”

For example, T cell receptors are present on T cells and are associated with CD3 molecules. T cell receptors are stimulated by antigen in the context of MHC molecules (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes, and expression of activation markers, e.g., CTLA-4 and/or CD28.

Transmission of a costimulatory signal to a T cell (e.g., via cross-linked CD28 molecules) involves a signaling pathway that is not inhibited by cyclosporin A. In addition, a costimulatory signal can induce cytokine secretion (e.g., IL-2 and/or IL-10) in a T cell and/or can prevent the induction of unresponsiveness to antigen, the induction of anergy, or the induction of cell death in the T cell.

As used herein, the term “inhibitory signal” refers to a signal transmitted via an inhibitory receptor (e.g., CTLA-4) on an immune cell. Such a signal antagonizes a signal transmitted via an activating receptor (e.g., via a TCR) and can result, e.g., in: inhibition of second messenger generation; inhibition of proliferation; inhibition of effector function in the immune cell, (e.g., reduced cellular cytotoxicity) the failure of the immune cell to produce mediators, (such as cytokines (e.g., IL-2) and/or mediators of allergic responses); or the development of anergy.

As used herein, the term “unresponsiveness” includes refractivity of immune cells to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory molecule) results in failure to produce cytokines and, thus, failure to proliferate. Anergic T cells can, however, mount responses to unrelated antigens and can proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. 1992. Science. 257:1134).

As used herein, the term “activity” with respect to a polypeptide includes activities which are inherent in the structure of a polypeptide. With respect to CTLA-4, the term “activity” includes the ability of a CTLA-4 polypeptide to bind to a costimulatory ligand (e.g., CD80 or CD86) and/or to modulate an inhibitory signal in an activated immune cell, e.g., by engaging a natural ligand on an antigen presenting cell. CTLA-4 transmits an inhibitory signal to a T cell. Modulation of an inhibitory signal in a T cell results in modulation of proliferation of and/or cytokine secretion by the T cell. CTLA-4 can also modulate a costimulatory signal by competing with a costimulatory receptor (e.g., CD28) for binding of costimulatory ligands. Thus, the term “CTLA-4 activity” includes the ability of a CTLA-4 polypeptide to bind its natural ligand(s), the ability to modulate immune cell costimulatory or inhibitory signals, and the ability to modulate the immune response. With respect to CD28, the term “activity” includes the ability of a CD28 polypeptide to bind to a costimulatory molecule (e.g., CD80 or CD86) and/or to modulate an activating signal in a naive or activate immune cell. CD28 transmits an activating co-stimulatory signal to a T cell. Modulation of an activating signal in a T cell results in increased proliferation of and/or cytokine secretion by the T cell. CD28 can also modulate a costimulatory signal by competing with an inhibitory receptor for binding of costimulatory molecules, e.g., CTLA-4. Thus, the term “CD28 activity” includes the ability of a CD28 polypeptide to bind to its natural ligand(s), the ability to modulate immune cell costimulatory or inhibitory signals, and the ability to modulate the immune response.

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The phrase “complementary determining region” (CDR) includes the region of an antibody molecule which comprises the antigen binding site.

The antibody may be an IgG such as IgGI, IgG2, IgG3 or IgG4; or IgM, IgA, IgE or IgD isotype. The constant domain of the antibody heavy chain may be selected depending upon the effector function desired. The light chain constant domain may be a kappa or lambda constant domain.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., hCTLA-4 or hCD28). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CHI domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (“scFv”); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

Antibodies may be polyclonal or monoclonal; xenogeneic, allogeneic, or syngeneic; or modified forms thereof, e.g. humanized, chimeric, etc. Preferably, antibodies of the invention bind specifically or substantially specifically to CTLA-4 or CD28 molecules present on a T cell of a subject. The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody composition, typically displays a single binding affinity for a particular antigen with which it immunoreacts.

The term “humanized antibody”, as used herein, is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences. Human antibodies are also within the scope of the invention.

An “isolated antibody”, as used herein, is intended to refer to an antibody that is substantially free of other antibodies having different antigenic specificities (e.g., an isolated antibody that specifically binds CTLA-4 or CD28 is substantially free of antibodies that specifically bind antigens other than CTLA-4 or CD28, respectively). Moreover, an isolated antibody may be substantially free of other cellular material and/or chemicals.

“Anti-CTLA-4 antibodies” are antibodies that specifically bind to a site on the extracellular domain of CTLA-4 protein, and transmit an inhibitory signal to a T cell. The term “anti-CTLA-4 antibodies” includes antibodies that block the binding of CTLA-4 to costimulatory ligands, e.g. CD80, CD86, etc. as well as those antibodies that do not block binding of CTLA-4 to costimulatory molecules. “Anti-CD28 antibodies” include antibodies that specifically bind to a site on the extracellular domain of CD28 protein, and which transmit an activating, costimulatory signal to a T cell. The term “anti-CD28 antibodies” includes antibodies that block the binding of CD28 to costimulatory ligands, e.g., CD80, CD86 and the line, as well as those antibodies that do not block binding of CD28 to costimulatory molecules.

The phrase “specifically” with reference to binding, recognition, or reactivity of antibodies includes antibodies which bind to naturally occurring molecules which are expressed transiently only on activated T cells. Specifically, with respect to CTLA-4, the term “specifically” with reference to binding, recognition, or reactivity of antibodies includes anti-CTLA-4 antibodies that bind to naturally occurring forms of CTLA-4, but are substantially unreactive with molecules related to CTLA-4, such as CD28 and other members of the immunoglobulin superfamily. With respect to CD28, the term “sepcifcially” with reference to binding, recognition, or reactivity of antibodies includes anti-CD28 antibodies that bind to naturally occurring forms of CD28, but are substantially unreactive with molecules related to CD28, such as CTLA-4 and other members of the immunoglobulin superfamily. Antibodies which are “substantially unreactive” with related molecules include antibodies which bind to CTLA-4 or CD28, but display no greater binding to molecules related to CTLA-4 or CD28 (but excluding CTLA-4 molecules, in the case of anti-CTLA4 antibodies, or CD28, in the case of anti-CD28 molecules) as compared to unrelated molecules, e.g., CD27. Preferably, anti-CTLA-4 or anti-CD28 antibodies bind to CTLA-4 or CD28, respectively, and bind to unrelated molecules or related molecules with only background binding. Antibodies specific for CTLA-4 from one source, e.g., human CTLA-4 or human CD28 may or may not be reactive with CTLA-4 or CD28 molecules from different species. Antibodies specific for naturally occurring CTLA-4 or CD28 may or may not bind to mutant forms of such molecules. In one embodiment, mutations in the amino acid sequence of a naturally occurring CTLA-4 or CD28 molecule result in modulation of the binding (e.g., either increased or decreased binding) of the antibody to the CTLA-4 or CD28 molecule. Antibodies to CTLA-4 or CD28 can be readily screened for their ability to meet this criteria. Assays to determine affinity and specificity of binding are known in the art, including competitive and non-competitive assays. Assays of interest include ELISA, RIA, flow cytometry, etc. Binding assays may use purified or semi-purified CTLA-4 or CD28 protein, or alternatively may use cells that express CTLA-4 or CD28, e.g. cells transfected with an expression construct for CTLA-4 or CD28; T cells that have been stimulated through cross-linking of CD3 and CD28; antigen and APCs; the addition of irradiated allogeneic cells, and the like. As an example of a binding assay, purified CTLA-4 or CD28 protein is bound to an insoluble support, e.g. microtiter plate, magnetic beads, etc. The candidate antibody and soluble, labeled CD80 or CD86 are added to the cells, and the unbound components are then washed off. The ability of the antibody to compete with CD80 and CD86 for CTLA-4 or CD28 binding is determined by quantitation of bound, labeled CD80 or CD86. Confirmation that the antibody binds specifically to CTLA-4 or CD28 can be confirmed by demonstrating that the antibody does not cross-react with CD28 or CTLA-4, respectively, using a similar assay, e.g., substituting CD28 for CTLA-4. An isolated antibody that specifically binds human CTLA-4 of CD28 may, however, have cross-reactivity to other antigens, such as CTLA-4 or CD28 molecules from other species.

As used herein, the term “causing to express” with reference to a construct includes art recognized methods by which a an exposed surface can be made to bear a particular molecule on that surface. For example, methods such as chemical cross-linking and transfection can be used to cause a surface to express a molecule of interest (e.g., an antigen binding portion of an anti-CTLA-4 or CD28 antibody or an MHC molecule).

For example, DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

As used herein, a “naturally-occurring” nucleic acid molecule refers to an RNA or DNA molecule having a nucleotide sequence that occurs in nature (e.g., encodes a natural protein).

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

As used herein, the term “vector” refers to a nucleic acid molecule capable of transporting another nucleic acid molecule to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “host cell” is intended to refer to a cell into which a nucleic acid molecule of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, an “isolated protein” refers to a protein that is substantially free of other proteins, cellular material and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the CTLA-4 or CD28 protein is derived, or substantially free from chemical precursors or other chemicals when chemically synthesized. The language “substantially free of cellular material” includes preparations of CTLA-4 or CD28 protein in which the protein is separated from cellular components of the cells from which it is isolated or recombinantly produced. In one embodiment, the language “substantially free of cellular material” includes preparations of CTLA-4 or CD28 protein having less than about 30% (by dry weight) of non-CTLA-4 or non-CD28 protein (also referred to herein as a “contaminating protein”), more preferably less than about 20% of non-CTLA-4 or non-CD28 protein, still more preferably less than about 10% of non-CTLA-4 or non-CD28 protein, and most preferably less than about 5% non-CTLA-4 or non-CD28 protein. When the CTLA-4 or CD28 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of CTLA-4 or CD28 protein in which the protein is separated from chemical precursors or other chemicals which are involved in the synthesis of the protein. In one embodiment, the language “substantially free of chemical precursors or other chemicals” includes preparations of CTLA-4 or CD28 protein having less than about 30% (by dry weight) of chemical precursors or non-CTLA-4 or non-CD28 chemicals, more preferably less than about 20% chemical precursors or non-CTLA-4 or non-CD28 chemicals, still more preferably less than about 10% chemical precursors or non-CTLA-4 or non-CD28 chemicals, and most preferably less than about 5% chemical precursors or non-CTLA-4 or non-CD28 chemicals.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid molecule and the amino acid sequence encoded by that nucleic acid molecule, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, |

CGT

| | |
|---|---|
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, |

TTG

| | |
|---|---|
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, |

TCT

| | |
|---|---|
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA molecule coding for a CTLA-4 or CD28 polypeptide or CTLA-4 or CD28 antibody of the invention (or any portion thereof) can be used to derive the CTLA-4 or CD28 polypeptide amino acid sequence or CTLA-4 or CD28 antibody amino acid sequence, using the genetic code to translate the CTLA-4 or CD28 polypeptide or CTLA-4 or CD28 antibody molecule into an amino acid sequence. Likewise, for any CTLA-4 or CD28 polypeptide or CTLA-4 or CD28 antibody—amino acid sequence, corresponding nucleotide sequences that can encode CTLA-4 or CD28 polypeptide or CTLA-4 or CD28 antibody protein can be deduced from the genetic code (which, because of its redundancy, will produce multiple nucleic acid sequences for any given amino acid sequence).

Thus, description and/or disclosure herein of a nucleotide sequence encoding a CTLA-4 or CD28 polypeptide or a nucleotide sequence encoding a CTLA-4 or CD28 antibody should be considered to also include description and/or disclosure of the amino acid sequence encoded by the nucleotide sequence. Similarly, description and/or disclosure of a CTLA-4 or CD28 polypeptide or CTLA-4 or CD28 antibody amino acid sequence herein should be considered to also include description and/or disclosure of all possible nucleotide sequences that can encode the amino acid sequence.

II. Preparation of Anti-CTLA-4 or Anti-CD28 Antibodies

A. Immunogens

One aspect of the invention pertains to anti-CTLA-4 or CD28 antibodies. Antibodies to CTLA-4 or CD28 can be made by immunizing a subject (e.g., a mammal) with a CTLA-4 or CD28 polypeptide or a nucleic acid molecule encoding a CTLA-4 or CD28 polypeptide or a portion thereof. In one embodiment, native CTLA-4 or CD28 proteins, or immunogenecic portions thereof, can be isolated from cells or tissue sources by an appropriate purification scheme using standard protein purification techniques. In another embodiment, CTLA-4 or CD28 proteins, or immunogenic portions thereof, can be produced by recombinant DNA techniques. Alternative to recombinant expression, a CTLA-4 or CD28 protein or immunogenic portion thereof, can be synthesized chemically using standard peptide synthesis techniques. Alternatively, nucleic acid molecules encoding a CTLA-4 or CD28 molecule or portion thereof can be used as immunogens. Whole cells expressing CTLA-4 or CD28 can be used as immunogens to produce anti-CTLA-4 or anti-CD28 antibodies.

The origin of the immunogen may be mouse, human, rat, monkey etc. The host animal will generally be a different species than the immunogen, e.g. mouse CTLA-4 or CD28 used to immunize hamsters, human CTLA-4 or CD28 to immunize mice, etc. The human and mouse CTLA-4 contain highly conserved stretches in the extracellular domain (Harper et al. (1991) J. Immunol. 147:1037-1044). Peptides derived from such highly conserved regions may be used as immunogens to generate cross-specific antibodies. The nucleotide and amino acid sequences of CTLA-4 from a variety of sources are known in the art. For example, the nucleotide and amino acid sequences of human CTLA-4 can be found in Dariavach et al. 1988. Eur. J. Immunol. 18:1901; Linsley et al. J. Exp. Med. 174:561; or Metzler et al. 1997. Nat. Struct. Biol. 4:525; or Harper et al. 1991. J. Immunol. 147:1037 or can be accessed on any of a variety of public or private databases, e.g., GenBank. Nucleotide and amino acid sequences encoding human CTLA-4 molecules are presented in SEQ ID NO: 1 and 2, respectively.

In one embodiment, the immunogen may comprise the complete protein, or fragments and derivatives thereof. Preferred immunogens comprise all or a part of the extracellular domain of human CTLA-4 (e.g., about amino acid residues 36-161 or about amino acids 38-161 of SEQ ID NO:2), where these residues contain the post-translation modifications, such as glycosylation, found on the native CTLA-4. Immunogens comprising the extracellular domain are produced in a variety of ways known in the art, e.g. expression of cloned genes using conventional recombinant methods, isolation from T cells, sorted cell populations expressing high levels of CTLA-4, etc. In another embodiment, the immunogen may comprise DNA encoding a CTLA-4 molecule or a portion thereof. For example, as set forth in the appended examples, 2 μg cDNA encoding the extracellular domain of recombinant human CTLA-4 could be used as an immunogen.

In a preferred embodiment, the immunogen is a human CTLA-4 molecule. Preferably, CTLA-4 proteins comprise the amino acid sequence encoded by SEQ ID NO: 1 or fragment thereof. In another preferred embodiment, the protein comprises the amino acid sequence of SEQ ID NO: 2 or fragment thereof. For example, the CTLA-4 molecule can differ in amino acid sequence from that shown in SEQ ID NO:2, e.g., can be from a different source or can be modified to increase its immunogenicity. In one embodiment, the protein has at least about 80%, and even more preferably, at least about 90% or 95% amino acid identity with the amino acid sequence shown in SEQ ID NO: 2.

Likewise, the nucleotide and amino acid sequences of CD28 from a variety of sources are known in the art. For example, the nucleotide and amino acid sequences of human CD28 can be found in the scientific literature (e.g., Aruffo A. and B. Seed. 1987. Proc. Natl. Acad. Sci. USA 84:8573; Gross, J. A. et al. 1990. J. Immunol. 144:3201; Clark, G J, and Dallman. 1992. Immunogenetics. 35:54) or can be accessed on any of a variety of public or private databases, e.g., GenBank. Nucleotide and amino acid sequences encoding human CD28 molecules are presented in SEQ ID NO:3 and 4, respectively.

In one embodiment, the immunogen may comprise the complete protein, or fragments and derivatives thereof. Preferred immunogens comprise all or a part of the extracellular domain of human CD28 (e.g., about amino acid residues 1 to about amino acid residue 134 of SEQ ID NO:4 or the sequence published in Aruffo and Seed, supra), where these residues contain the post-translation modifications, such as glycosylation, found on the native CD28. Immunogens comprising the extracellular domain are produced in a variety of ways known in the art, e.g. expression of cloned genes using conventional recombinant methods, isolation from T cells, sorted cell populations expressing high levels of CD28, etc. In another embodiment, the immunogen may comprise DNA encoding a CD28 molecule or a portion thereof. For example, cDNA, e.g., encoding the extracellular domain of recombinant human CD28 could be used as an immunogen.

In a preferred embodiment, the immunogen is a human CD28 molecule. Preferably, CD28 proteins comprise the amino acid sequence encoded by SEQ ID NO:3 or fragment thereof. In another preferred embodiment, the protein comprises the amino acid sequence of SEQ ID NO: 4 or fragment thereof. For example, the CD28 molecule can differ in amino acid sequence from that shown in SEQ ID NO:4, e.g., can be from a different source or can be modified to increase its immunogenicity. In one embodiment, the protein has at least about 80%, and even more preferably, at least about 90% or 95% amino acid identity with the amino acid sequence shown in SEQ ID NO: 4.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100). The percent identity between the two sequences is a function of the number of identical positions shared by the sequences, taking into account the number of gaps, and the length of each gap, which need to be introduced for optimal alignment of the two sequences. As used herein amino acid or nucleic acid “identity” is equivalent to amino acid or nucleic acid “homology”.

The comparison of sequences and determination of percent identity between two sequences can be accomplished using a mathematical algorithm. In a preferred embodiment, the percent identity between two amino acid sequences is determined using the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6.

The nucleic acid and protein sequences of the CTLA-4 or CD28 can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to CTLA-4 nucleic acid molecules of the invention. BLAST protein searches can be performed with the XBLAST program, score=50, wordlength=3 to obtain amino acid sequences homologous to CTLA-4 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. For example, the nucleotide sequences of the invention were analyzed using the default Blastn matrix 1-3 with gap penalties set at: existence 11 and extension 1. The amino acid sequences of the invention were analyzed using the default settings: the Blosum62 matrix with gap penalties set at existence 11 and extension 1. See http://www.ncbi.nlm.nih.gov.

CTLA-4 or CD28 chimeric or fusion proteins or nucleic acid molecules encoding them can also be used as immunogens. As used herein, a CTLA-4 or CD28 “chimeric protein” or “fusion protein” comprises a CTLA-4 or CD28 polypeptide operatively linked to a non-CTLA-4 or non-CD28 polypeptide. A “CTLA-4 polypeptide” or “CD28 polypeptide” refers to a polypeptide having an amino acid sequence corresponding to CTLA-4 polypeptide, whereas a “non-CTLA-4 polypeptide” or “non-CD28 polypeptide” refers to a polypeptide having an amino acid sequence corresponding to a protein which is not substantially homologous to the CTLA-4 or CD28 protein, e.g., a protein which is different from the CTLA-4 or CD28 protein and which is derived from the same or a different organism. Within a CTLA-4 or CD28 fusion protein the CTLA-4 or CD28 polypeptide can correspond to all or a portion of a CTLA-4 or CD28 protein. In a preferred embodiment, a CTLA-4 or CD28 fusion protein comprises at least one biologically active portion of a CTLA-4 or CD28 protein, e.g., an extracellular domain of a CTLA-4 or CD28 protein. Within the fusion protein, the term “operatively linked” is intended to indicate that the CTLA-4 or CD28 polypeptide and the non-CTLA-4 or non-CD28 polypeptide are fused in-frame to each other. The non-CTLA-4 or non-CD28 polypeptide can be fused to the N-terminus or C-terminus of the CTLA-4 or CD28 polypeptide.

Preferably, a CTLA-4 or CD28 fusion protein or nucleic acid molecule encoding a CTLA-4 or CD28 fusion protein is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers.

Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide or an HA epitope tag). A CTLA-4 or CD28 encoding nucleic acid can be cloned into such an expression vector such that the fusion moiety is linked in-frame to the CTLA-4 or CD28 protein. Such fusion moieties can be linked to the C or to the N terminus of the CTLA-4 or CD28 protein or a portion thereof.

Variants of the CTLA-4 or CD28 proteins can also be generated by mutagenesis, e.g., discrete point mutation or truncation of a CTLA-4 or CD28 protein and used as a immunogen. In one embodiment, variants of a CTLA-4 or CD28 protein can be identified by screening combinatorial libraries of mutants, e.g., truncation mutants, of a CTLA-4 or CD28 protein for CTLA-4 or CD28 protein agonist or antagonist activity. In one embodiment, a variegated library of CTLA-4 or CD28 variants is generated by combinatorial mutagenesis at the nucleic acid level and is encoded by a variegated gene library. A variegated library of CTLA-4 or CD28 variants can be produced by, for example, enzymatically ligating a mixture of synthetic oligonucleotides into gene sequences such that a degenerate set of potential CTLA-4 or CD28 sequences is expressible as individual polypeptides, or alternatively, as a set of larger fusion proteins (e.g., for phage display) containing the set of CTLA-4 or CD28 sequences therein. There are a variety of methods which can be used to produce libraries of potential CTLA-4 or CD28 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CTLA-4 or CD28 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a CTLA-4 or CD28 protein coding sequence can be used to generate a variegated population of CTLA-4 or CD28 fragments for screening and subsequent selection of variants of a CTLA-4 or CD28 protein. In one embodiment, a library of coding sequence fragments can be generated by treating a double stranded PCR fragment of a CTLA-4 or CD28 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the CTLA-4 or CD28 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CTLA-4 or CD28 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify CTLA-4 or CD28 variants (Arkin and Yourvan (1992) Proc. Natl. Acad. Sci. USA 89:7811-7815; Delgrave et al. (1993) Protein Engineering 6(3):327-331).

In one embodiment, cell based assays can be exploited to analyze a variegated CTLA-4 or CD28 library. For example, a library of expression vectors can be transfected into a cell line which ordinarily synthesizes and secretes CTLA-4 or CD28. The transfected cells are then cultured such that CTLA-4 or CD28 and a particular mutant CTLA-4 or CD28 are secreted and the effect of expression of the mutant on CTLA-4 or CD28 activity in cell supernatants can be detected, e.g., by any of a number of enzymatic assays. Plasmid DNA can then be recovered from the cells which score for inhibition, or alternatively, potentiation of CTLA-4 or CD28 activity, and the individual clones further characterized.

An isolated CTLA-4 or CD28 protein, or a portion or fragment thereof, or nucleic acid molecules encoding a CTLA-4 or CD28 polypeptide of portion thereof, can be used as an immunogen to generate antibodies that bind CTLA-4 or CD28 using standard techniques for polyclonal and monoclonal antibody preparation. In one embodiment, a full-length CTLA-4 or CD28 protein or nucleic acid molecule encoding a full-length CTLA-4 or CD28 protein can be used. Alternatively, an antigenic peptide fragment (i.e., a fragment capable of promoting an antigenic response) of a CTLA-4 or CD28 polypeptide or nucleic acid molecule encoding a fragment of a CTLA-4 or CD28 polypeptide can be used can be used as the immunogen. An antigenic peptide fragment of a CTLA-4 or CD28 polypeptide typically comprises at least 8 amino acid residues (e.g., at least 8 amino acid residues of the amino acid sequence shown in SEQ ID NO: 2 or SEQ ID NO:4) and encompasses an epitope of a CTLA-4 or CD28 polypeptide such that an antibody raised against the peptide forms an immune complex with a CTLA-4 or CD28 molecule. Preferred epitopes encompassed by the antigenic peptide are regions of CTLA-4 or CD28 that are located on the surface of the protein, e.g., hydrophilic regions. In another embodiment, an antibody binds specifically to a CTLA-4 or CD28 polypeptide. In a preferred embodiment, the CTLA-4 or CD28 polypeptide is a human CTLA-4 or CD28 polypeptide.

Preferably, the antigenic peptide comprises at least about 10 amino acid residues, more preferably at least about 15 amino acid residues, even more preferably at least 20 about amino acid residues, and most preferably at least about 30 amino acid residues. Preferred epitopes encompassed by the antigenic peptide are regions of a CTLA-4 or CD28 polypeptide that are located on the surface of the protein, e.g., hydrophilic regions, and that are unique to a CTLA-4 or CD28 polypeptide. In one embodiment such epitopes can be specific for a CTLA-4 or CD28 proteins from one species, such as mouse or human (i.e., an antigenic peptide that spans a region of a CTLA-4 or CD28 polypeptide that is not conserved across species is used as immunogen; such non conserved residues can be determined using an amino acid sequence, e.g., using one of the programs described supra). A standard hydrophobicity analysis of the CTLA-4 or CD28 protein can be performed to identify hydrophilic regions.

A CTLA-4 or CD28 immunogen typically is used to prepare antibodies by immunizing a suitable subject, (e.g., rabbit, goat, mouse or other mammal) with the immunogen. An appropriate immunogenic preparation can contain, for example, a nucleic acid molecule encoding a CTLA-4 or CD28 immunogen, a recombinantly expressed CTLA- or CD28 protein or a chemically synthesized CTLA-4 or CD28 immunogen. The preparation can further include an adjuvant, such as Freund's complete or incomplete adjuvant, alum, a cytokine or cytokines, or similar immunostimulatory agent. Immunization of a suitable subject with an immunogenic CTLA-4 or CD28 preparation induces a polyclonal anti-CTLA-4 or CD28 antibody response.

B. Anti-CTLA-4 or Anti-CD28 Antibodies

Antibodies typically comprise two heavy chains linked together by disulphide bonds and two light chains. Each light chain is linked to a respective heavy chain by disulphide bonds. Each heavy chain has at one end a variable domain followed by a number of constant domains. Each light chain has a variable domain at one end and a constant domain at its other end. The light chain variable domain is aligned with the variable domain of the heavy chain. The light chain constant domain is aligned with the first constant domain of the heavy chain. The constant domains in the light and heavy chains are not involved directly in binding the antibody to antigen. The variable domains of each pair of light and heavy chains form the antigen binding site.

The domains on the light and heavy chains have the same general structure and each domain comprises a framework of four regions, whose sequences are relatively conserved, connected by three complementarity determining regions (CDRs). The four framework regions largely adopt a beta-sheet conformation and the CDRs form loops connecting, and in some cases forming part of, the beta-sheet structure. The CDRs are held in close proximity by the framework regions and, with the CDRs from the other domain, contribute to the formation of the antigen binding site. CDRs and framework regions of antibodies may be determined by reference to Kabat et al (“Sequences of proteins of immunological interest” US Dept. of Health and Human Services, US Government Printing Office, 1987).

Polyclonal anti-CTLA-4 or CD28 antibodies can be prepared as described above by immunizing a suitable subject with a CTLA-4 or CD28 immunogen. The anti-CTLA-4 or anti-CD28 antibody titer in the immunized subject can be monitored over time by standard techniques, such as with an enzyme linked immunosorbent assay (ELISA) using immobilized a CTLA-4 or CD28 polypeptide. If desired, the antibody molecules directed against a CTLA-4 or CD28 polypeptide can be isolated from the mammal (e.g., from the blood) and further purified by well known techniques, such as protein A chromatography to obtain the IgG fraction. At an appropriate time after immunization, e.g., when the anti-CTLA-4 or CD28 antibody titers are highest, antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol 127:539-46; Brown et al. (1980) J. Biol Chem 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet., 3:231-36). Briefly, an immortal cell line (typically a myeloma) is fused to lymphocytes (typically splenocytes) from a mammal immunized with a CTLA-4 or CD28 immunogen as described above, and the culture supernatants of the resulting hybridoma cells are screened to identify a hybridoma producing a monoclonal antibody that binds specifically to a CTLA-4 or CD28 polypeptide.

Any of the many well known protocols used for fusing lymphocytes and immortalized cell lines can be applied for the purpose of generating an anti-CTLA-4 or CD28 monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a CTLA-4 or CD28 molecule, e.g., using a standard ELISA assay.

As an alternative to preparing monoclonal antibody-secreting hybridomas, a monoclonal anti-CTLA-4 or anti-CD28 antibody can be identified and isolated by screening a recombinant combinatorial immunoglobulin library (e.g., an antibody phage display library) with or CD28CTLA-4 (or a portion of a or CD28CTLA-4 molecule, e.g., the extracellular domain of or CD28CTLA-4) to thereby isolate immunoglobulin library members that bind a CTLA-4 polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J. Mol Biol 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; Barbas et al. (1991) PNAS 88:7978-7982; and McCafferty et al. Nature (1990) 348:552-554.

Anti-CTLA-4 or anti-CD28 antibodies may bind to any portion of the CTLA-4 or CD28 molecule such that, in the case of CTLA-4, an inhibitory signal is transmitted upon the binding of the antibody to CTLA-4, or, in the case of CD28, a costimulatory signal is transmitted upon the binding of the antibody to CD28. Preferably, anti-CTLA-4 or anti-D28 antibodies bind to the extracellular domain of the CTLA-4 or CD28 molecule. Preferred anti-CTLA-4 or CD28 antibodies bind to a CTLA-4 or CD28 molecule in the subject to which the antibodies (or constructs bearing the antibodies) will be administered or in which the antibodies (or construct bearing the antibodies) will be expressed An exemplary anti-CTLA-4 or CD28 antibody for use in the instant invention is the anti-human CTLA-4 or CD28 antibody made in a rodent.

C. Alteration of Antibodies

A variety of different alterations or changes can be introduced into the subject antibodies to optimize their use in downmodulating the immune response. For example, mutations can be introduced into constant and/or variable regions to preserve or enhance e.g., affinity, specificity, and/or half life optionally, alteration may be introduced to decrease immunogenicity. For example, conservative amino acid substitutions can be made. Exemplary changes include: substitution of isoleucine, valine, and leucine for any other of these hydrophoic amino acids. Aspartic acid can be substituted for glutamic acid and vice versa. Glutamine can be substituted for asparagine and vice versa. Serine can be substituted for threonine and vice versa. Other substitutions can also be considered to be conservative, depending on the environment of the particular amino acid and its role in the three-dimensional structure of the protein. For example, glycine and alanine can be interchangeable, as can alanine and valine. Methionine, which is relatively hydrophobic, can often be interchanged with leucine and isoleucine, and sometimes with valine. Lysine and arginine can be interchangeable in locations in which the significant feature of the amino acid residue is its charge and the differing pK's of the two amino acid residues are not significant. Changes that do not affect the three-dimensional structure or the reactivity of the protein can be determined by computer modeling.

For in vivo use, particularly for injection into humans, it is often desirable to decrease the antigenicity of an antibody. An immune response of a recipient against the blocking agent will potentially decrease the period of time that the therapy is effective. To minimize such an immune response, humanized or chimeric antibodies can be constructed. Various methods of humanizing antibodies can be used. For example, the humanized antibody may be the product of an animal having transgenic human immunoglobulin constant region genes (see for example International Patent Applications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) P.N.A.S. 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Additionally, recombinant anti-CTLA-4 or anti-CD28 ntibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060. In addition, humanized antibodies can be made according to standard protocols such as those disclosed in U.S. Pat. Nos. 5,777,085; 5,530,101; 5,693,762; 5,693,761; 5,882,644; 5834597; 5932448; or 5,565,332.

For example, an antibody may be humanized by grafting the desired CDRs onto a human framework, e.g., according to EP-A-0239400. A DNA sequence encoding the desired reshaped antibody can be made beginning with the human DNA whose CDRs it is wished to reshape. The rodent variable domain amino acid sequence containing the desired CDRs is compared to that of the chosen human antibody variable domain sequence. The residues in the human variable domain are marked that need to be changed to the corresponding residue in the rodent to make the human variable region incorporate the rodent CDRs. There may also be residues that need substituting, e.g., adding to or deleting from the human sequence. Oligonucleotides can be synthesized that can be used to mutagenize the human variable domain framework to contain the desired residues. Those oligonucleotides can be of any convenient size.

Alternatively, humanization may be achieved using the recombinant polymerase chain reaction (PCR) methodology taught, e.g., in WO 92/07075. Using this methodology, a CDR may be spliced between the framework regions of a human antibody. In general, the technique of WO 92/07075 can be performed using a template comprising two human framework regions, AB and CD, and between them, the CDR which is to be replaced by a donor CDR. Primers A and B are used to amplify the framework region AB, and primers C and D used to amplify the framework region CD. However, the primers B and C each also contain, at their 5′ ends, an additional sequence corresponding to all or at least part of the donor CDR sequence. Primers B and C overlap by a length sufficient to permit annealing of their 5′ ends to each other under conditions which allow a PCR to be performed. Thus, the amplified regions AB and CD may undergo gene splicing by overlap extension to produce the humanized product in a single reaction.

D. Construction of scFv Antigen Binding Portions of Anti-CTLA-4 or Anti-CD28

Single-chain Fv (ScFv) molecules are antibody binding portions in which the VH and VL partner domains are linked via a flexible oligopeptide. Methods of making scFv molecules are known in the art. (Bird et al (1988) Science 240, 423; Huston et al (1988) Proc. Natl. Acad. Sci, USA 85, 5879).

For example, MRNA can be isolated from hybridoma cells producing anti-CTLA-4 or anti-CD28. Typically, total RNA is isolated by extraction methods well known in the art, such as extraction with phenol at acid pH or extraction with guanidinium thiocyanate followed by centrifugation in cesium chloride solutions. These procedures, and others for RNA extraction, are disclosed in J. Sambrook et al., “Molecular Cloning: A Laboratory Manual” (2d ed., Cold Spring Harbor Laboratory Press, Cold Spring Harbor, N.Y., 1989), ch. 7, “Extraction, Purification, and Analysis of Messenger RNA From Eukaryotic Cells,” pp. 7.1-7.25. Optionally, the mRNA can be isolated from the total mRNA by chromatography on oligo (dT) cellulose, but this step is not required.

To synthesize cDNA, primers complementary to the κ or λ light chain constant region and to the constant region of the y2a heavy chain are preferably used to initiate synthesis. Amplification can be carried out by any procedure allowing high fidelity amplification without slippage. Preferably, amplification is by the polymerase chain reaction procedure (K. B. Mullis & F. A. Faloona, “Specific Synthesis of DNA in Vitro Via a Polymerase-Catalyzed Chain Reaction,” Meth. Enzymol. 155:335-350 (1987); K. Mullis et al., “Specific Enzymatic Amplification of DNA in Vitro: The Polymerase Chain Reaction,” Cold Spring Harbor Symp. Quant. Biol. 51:263-273 (1986); R. K. Saiki et al., “Primer-Directed Enzymatic Amplification of DNA with a Thermostable DNA Polymerase,” Science 238:487-491 (1988)).

One highly preferred procedure uses singlesided or anchored PCR (E. Y. Loh et al., “Polymerase Chain Reaction with Single-Sided Specificity: Analysis of T-cell Receptor δ Chain,” Science 243:217-220 (1989)). This procedure uses homopolymer tailing of the 3′-end of the reverse transcript; PCR amplification is then performed with a specific 3′-primer and a second oligonucleotide consisting of a homopolymer tail complementary to the homopolymer tail added to the 3′-end of the transcript attached to a sequence with a convenient restriction site, termed the anchor. One version is described in (Y. L. Chiang et al., “Direct cDNA Cloning of the Rearranged Immunoglobulin Variable Region,” Biotechniques 7:360-366 (1989)).

The PCR products are cloned into a suitable host, e.g., E. coli. A number of cloning vectors suitable for cloning into E. coli are known and are described in vol. 1 of Sambrook et al., supra, Ch. 1, “Plasmid Vectors,” pp. 1.1-1.110. The exact manipulations required depend on the particular cloning vector chosen and on the particular restriction endonuclease sites used for cloning into the vector. One highly preferred vector is pUC19. For cloning into pUC 19, the PCR products are treated with the Klenow fragment of E. coli DNA polymerase I and with the four deoxyribonucleoside triphosphates to obtain blunt ends by filling single-stranded regions at the end of the DNA chains. PCR can then be used to add Eco RI and Bam HI restriction sites to the 5′-end and 3′-ends, respectively, of the amplified fragment of cDNA of light-chain origin (the VL fragment). Similarly, Xba I and Hind III restriction sites are added to the amplified fragment of cDNA of heavy chain origin (the VH fragment). The fragments are digested with the appropriate restriction endonucleases and are cloned into pUC19 vector that had been digested with: (1) Eco RI and Bam HI for VL and (2) Xba I and Hind III for VH. The resulting constructs can be used to transform a competent cell, e.g., an E. coli strain.

Clones containing VL and VH are preferably identified by DNA sequencing. A suitable DNA sequencing procedure is the Sanger dideoxynucleotide chain termination procedure. Such a procedure can be performed using the Sequenase 2.0 kit (United States Biochemical, Cleveland, Ohio), with forward and reverse primers that anneal to the pUC19 sequences flanking the polycloning site. Preferably, consensus sequences for VL and VH are determined by comparing multiple clones and aligning the sequences with corresponding murine VL and VH variable region sequences (E. A. Kabat et al., “Sequences of Proteins of Immunological Interest” (4th ed., U.S. Department of Health and Human Services, Bethesda, Md., 1987)).

Clones containing VL and VH sequences can be placed in an expression cassette incorporating a single-chain antibody construct including the VL and VH sequences separated by a linker. In one highly preferred procedure, the 5′-leader sequence is removed from VL and replaced with a sequence containing a Sal I site preceding residue 1 of the native protein. Constant region residues from the 3′-end are replaced with a primer adding a sequence complementary to a sequence coding for a linker sequence (e.g., the 16-residue linker sequence ESGSVSSEELAFRSLD (J. K. Batra et al., “Anti-TAc (FV)-PE40, a Single Chain Antibody Pseudomonas Fusion Protein Directed at Interleukin-2 Receptor-Bearing Cells,” J. Biol. Chem. 265:15198-15202 (1990)).

For the VH sequence, a VH primer adds the “sense” sequence encoding the linker, e.g., the 16-residue linker sequence given above to the VH 5′-end preceding residue 1 of the mature protein and substitutes a Bcl I site for the constant region residues at the 3′-end.

The polymerase chain reaction can then be used with a mixture of VL and VH cDNA, as templates, and a mixture of the four primers (two linker primers and two primers containing restriction sites). This creates a single DNA fragment containing a VL-linker-VH sequence flanked by Sal I and Bcl I sites. The DNA construct is then preferably passaged through, e.g., E. coli cells. The passaged construct is then digested with Sal I and Bcl I.

For preparation and expression of the fusion protein, digested DNA from the preceding step is then ligated into a pCDM8 vector containing the anti-L6 K light chain leader sequence followed by a Sal I site and a Bcl I site preceding cDNA encoding a human IgG1 tail in which cysteines in the hinge region are mutated to serines to inhibit dimerization (P. S. Linsley et al., “Binding of the B Cell Activation Antigen B7 to CD28 Costimulates T-Cell Proliferation and Interleukin-2 mRNA Accumulation,” J. Exp. Med. 191:721-730 (1991)).

The resulting construct is capable of expressing anti-CTAL4 scFv chimeric humanized monoclonal antibody. Preferred constructs comprise murine CDRs and human constant regions.

Plasmid DNA can then isolated and purified, such as by cesium chloride density gradient centrifugation. The purified DNA is then transfected, preferably into a eukaryotic cell line, capable of expressing such transfected DNA. A highly preferred cell line is monkey COS cells. A preferred method of introducing DNA is by DEAE-dextran, but other methods are known in the art. These methods include contacting a cell with coprecipitates of calcium phosphate and DNA, use of a polycation, polybrene, or electroporation. These methods are described in J. Sambrook et al., “Molecular Cloning: A Laboratory Manual,” supra, vol. 3, pp. 16.30-16.55.

Preferably, recombinant DNA containing the sequence coding for the fusion protein is expressed by transient expression, as described in A. Aruffo, “Transient Expression of Proteins Using COS Cells,” in Current Protocols in Molecular Biology (2d ed., F. M. Ausubel et al., eds., John Wiley & Sons, New York, 1991), pp. 16.13.1-16.13.7.

III. MHC Molecules

MHC molecules for use in connection with the instant invention include both class I and class II molecules. Class I molecules include, but are not limited to, different antigenic specificities of HLA-A, B, and C class I proteins. Different antigenic specificities of HLA-DR, HLA-DQ, HLA-DP, and HLA-DW class II proteins can also be used (WHO Nomenclature Committed, Immunogenetices 16:135 (1992); Hensen et al., in “Fundamental Immunology,” ed. W. Paul, pp. 577-628, Raven Press, New York, 1993; and see NIH Genbank and EMBL data bases for HLA protein sequences).

MHC molecules are usually occupied with peptides, e.g., peptides that have been processed by the cell that is presenting them. Accordingly, in one embodiment, for downmodulation of an undesirable immune response in a subject, it may be desirable to employ empty MHC molecules in a construct of the invention to facilitate loading the molecules with a peptide to which an immune response in a subject is directed.

Two pathways are thought to exist within vertebrate cells to generate peptides for recognition by T cells. One is the endogenous pathway, which processes endogenously expressed antigenic proteins and provides peptides to MHC class I molecules for antigen presentation to CD8+ T cells. This process involves proteasomes and the ubiquitin pathway of protein degradation. The other is the exogenous pathway, which processes exogenous antigenic proteins and provides peptides to HLA Class II molecules for presentation to CD4+T cells.

MHC molecules can be dissociated from peptides, e.g., using a mild acid treatment and associating selected peptides with the MHC molecule (e.g., U.S. Pat. No. 5,846,827). Empty MHC molecules can be made to bind to a peptide to which an immune response sought to be downmodulated is specific, e.g., by loading the constructs bearing MHC molecules in culture e.g., Tykocinski et al., Amer. J. Pathol. 148:1-16 (1996). Peptide or protein pulsing (co-culturing) may also be used (Inaba et al., J. Exp. Med. 172:631-640 (1990)).

Alternatively, molecules may be introduced to surfaces via fusion with liposomes bearing the selected antigen molecules (Coeshott et al., J. Immunol. 134:1343-1348 (1985)). Cell fusion techniques include those in which antigen bearing cells are fused with constructs to introduce the desired target antigen into the construct (Guo et al., Science 263:518-520 (1994)).

Alternatively, genetic material encoding selected antigens may be introduced into cells and the cellular processing machinery can be employed to express desired peptides in the context of the appropriate MHC molecule.

IV. Preparation of Agents For Downmodulating (anti-CTLA4) or Upmodulating (anti-CD28) The Immune Response

A. Surfaces

Constructs for downmodulating the immune response in a subject comprise a surface that, upon introduction into a subject, would be exposed to the extracellular milieu. It is to this external, exposed surface to which an antigen binding portion of an antibody that binds CTLA-4 or CD28 and an MHC molecule are attached to make a construct of the invention. In this manner, the antigen binding portion of the antibody and the MHC molecule are available to bind to the appropriate molecules expressed on a T cell of the subject to which the constructs are administered.

Constructs of the invention comprise a surface which acts to anchor an antigen-binding portion of an antibody that binds to CTLA-4 or CD28 and an MHC molecule. A variety of different surfaces can be used in making the constructs of the invention. For example, in one embodiment, antibody binding portions can be attached to polymers comprising an exposed surface. Exemplary polymers include polysaccharides, acrylic polymers (e.g., polyacrolein or polystyrene or poly (alpha-hydroxy acids)), lactic acid polymers, or copolymers such as, polymers of lactic and glycolic acids. Beads and microbeads comprising a surface to which antigen binding portions of anti-CTLA-4 or anti-CD28 antibody and MHC molecules can be attached are known in the art (see, e.g., U.S. Pat. No. 5,871,747 and the like).

In another embodiment, the construct comprising a surface comprises a lipid bilayer. For example, such a construct can be an acellular construct, e.g., a liposome or a micelle. In yet another embodiment, a construct for use in the instant invention is a cell, such as a prokaryotic or a eukaryotic cell. Cells may be derived from any tissue or organ. Exemplary cells are derived from peripheral blood. Preferred cells include cells that can be maintained in culture.

In one embodiment, a cell for use in a construct of the invention is a syngeneic cell. In another embodiment, a cell for use in a construct of the invention is an allogeneic cell. In yet another embodiment, a cell for use in a construct of the invention is a xenogeneic cell. In one embodiment, e.g., when the cell is an allogeneic or a xenogenic cell, the cell is selected to provide a missing or diminished function in the subject. For example, in the case of a subject that would benefit from transplantation of a liver cell, a liver cell is used in the subject construct.

A cell for use in a construct of the invention can be a wild-type (naturally occurring) cell or can comprise alterations that optimize its use in the subject constructs. In another embodiment, such a cell can be altered to express molecules which enhance its ability to bind to a T cell in a subject, e.g., by altering the cell to express adhesion molecules. For example, such a cell can be altered to eliminate expression of molecules that promote immunostimulation (e.g., CD28 or cytokines). In another example, such a cell can be altered to modify the ability of such a cell to process antigen so that the peptides presented by the cell can be controlled (e.g., by introducing a defect in antigen processing, see e.g., U.S. Pat. No. 5,731,160).

B. Methods of Attaching Molecules to Surfaces

Surface-bound molecules (e.g., anti-CTLA-4 or anti-CD28 molecules and MHC molecules) can be attached to an exposed surface using a variety of art-recognized techniques, e.g., U.S. Pat. No. 6,046,173. For example, in one embodiment a molecule for attachment can be associated with the exposed surface of the construct, e.g., in a covalent linkage. Covalent linkage includes, e.g., linkage by a bifunctional coupling agent and oxidative linkage. In one embodiment a molecule for attachment can be attached to the exposed surface directly (e.g., to the surface itself). In another embodiment, a molecule can be attached indirectly (e.g., attached to another molecule, such as a lipid (e.g., a fatty acid chain or prenyl group) or a polypeptide, present on the exposed surface.

Many bifunctional coupling agents are useful for coupling organic molecules possessing various types of functional groups to proteins, including antibody molecules. The conjugation of organic molecules to proteins, including proteins possessing antibody specificity, is well-known in the art and is described, for example, in P. Tijssen, “Practice and Theory of Enzyme Immunoassays” (Elsevier, Amsterdam, 1985), pp. 2/9-296, incorporated herein by reference.

Briefly, organic molecules containing carboxyl groups or that can be carboxylated, can be coupled by the mixed anhydride reaction, by reaction with a water-soluble carbodiimide, or esterification with N-hydroxysuccinimide. Carboxylation can be performed by reactions such as alkylation of oxygen or nitrogen substituents with haloesters, followed by hydrolysis of the ester, or the formation of hemisuccinate esters or carboxymethyloximes on hydroxyl or ketone groups of steroids.

Organic molecules with amino groups or nitro groups reducible to amino groups can be converted to diazonium salts and reacted with proteins at mildly alkaline pH, for aromatic amines. Organic molecules with aliphatic amines can be conjugated to proteins by various methods, including reaction with carbodiimides, reaction with the homobifunctional reagent tolylene-2,4-diisocyanate, or reaction with maleimide compounds. Aliphatic amines can also be converted to aromatic amines by reaction with p-nitrobenzoylchloride and subsequent reduction to a p-aminobenzoylamide, which can then be coupled to proteins after diazotization. Also, bifunctional imidate esters, such as dimethylpimelimidate, dimethyladipimidate, or dimethylsuberimidate, can be used to conjugate amino group-containing organic molecules to proteins.

Thiol-containing organic molecules can be conjugated to proteins with malemides, such as 4-(N-maleimidomethyl)-cyclohexane-1-carboxylic acid N-hydroxysuccinimide ester.

For organic molecules with hydroxyl groups, an alcohol function can be converted to the hemisuccinate, which introduces a carboxyl group available for conjugation. Alternatively, the bifunctional reagent sebacoyldichloride converts an alcohol to an acid chloride, which then reacts with proteins.

Phenols can be activated with diazotized p-aminobenzoic acid, which introduces a =carboxyl group, and can then be reacted with the proteins by the mixed anhydride reaction. Sugars can be activated by forming a p-nitrophenyl glycoside, followed by reduction of the nitro group to an amino group and conjugation by diazotization. Other methods include the cleavage of vicinal glycols of sugars to aldehydes by reaction with periodate, followed by coupling to amines by reductive alkylation with sodium borohydride. Alternatively, hydroxyl containing organic molecules can be conjugated after conversion to chlorocarbonates by reaction with phosgene.

For organic molecules with aldehyde or ketone groups, carboxyl groups can be introduced through the formation of O-carboxymethyloximes. Ketone groups can also be derivatized with p-hydrazinobenzoic acid to produce carboxyl groups.

Organic molecules containing aldehydes can be directly conjugated through the formation of Schiff bases that are stabilized by reaction with a reducing agent such as sodium borohydride.

Oxidative linkages can also be used. Oxidative linkage is particularly useful when coupling radioactive iodine to antibodies. Suitable methods include: (1) chemical oxidation with chloramine-T; (2) chemical oxidation with iodogen (1,3,4,6-tetrachloro 3.alpha.,6.alpha.-diphenylglycoluril); (3) oxidation with the enzyme lactoperoxidase. Although not an oxidative procedure, another useful method for labeling with iodine is with ¹²⁵I N-succinimidyl 3-(4-hydroxyphenylpropionate), generally known as Bolton-Hunter reagent. These techniques are described, e.g., in E. Harlow and D. Lane, “Antibodies: A Laboratory Manual” (Cold Spring Harbor Laboratory, Cold Spring Harbor, N.Y., 1988), pp. 324-339.

In another embodiment, such a molecule can be attached via a transmembrane domain (e.g., a membrane-spanning region of an integral membrane protein). Such a domain can be derived from the protein to be attached or from a different protein. For example, integral membrane proteins from which transmembrane domains can be derived include cell surface receptors (e.g., growth factor receptors), adhesion molecules (e.g., integrins, or selectins), or CD antigens. For example, transmembrane domains can be Type I domains which comprise about 25 hydrophobic amino acid residues and are usually followed by a cluster of basic amino acids (e.g., as found in CD2, CD40, or IL-4 receptor). Type II transmembrane domains can also be used. Type II domains cross the membrane such that the carboxy-terminal portion of the polypeptide is extracellular (e.g., in the case of CD72). Type III transmembrane domains can also be employed. Such domains cross a lipid bilayer numerous times (e.g., as in the case of G-protein linked receptors).

In another embodiment, a molecule can be attached to the subject constructs using a glycosylphosphatidylinositol (GPI) anchor attached to the carboxy-terminal residue of the molecule. For example, GPI anchors can be derived from human placental alkaline phosphatase (see, e.g., Whitehorn et al. 1995 Biotechnology 13:1215-1219). GPI anchored molecules may have a signal sequence at their carboxy-terminus that is cleaved off and replaced by the GPI anchor (see, e.g., U.S. Pat. No. 5,891,432).

C. Alteration of Cells to Express Surface-Bound Molecules

Any of a variety of different methods can be used to cause a cell to express an active molecule of the invention (e.g., an anti-CTLA-4 or anti-CD28 molecule and/or an MHC molecule. For example, as set forth above, active molecules can be linked to cells as well as other surfaces to form the constructs of the invention. In addition, cells can be caused to express an active molecule by various nucleic acid manipulation procedures.

Techniques for nucleic acid manipulation are well known. (See, e.g., Sambrook et al., (1989); Ausubel et al. (1987) and in Annual Reviews of Biochemistry, 61:131-156 (1992)). Reagents useful in applying such techniques, such as restriction enzymes and the like, are widely known in the art and commercially available from a number of vendors.

Nucleic acid sequences encoding the selected molecules for expression in the invention may be obtained using known procedures for molecular cloning and replication of the vector or plasmid carrying the sequences in a suitable host cell.

Nucleic acid sequences for use in the present invention may also be produced in part or in total by chemical synthesis, e.g. by the phosphoramidite method described by Beaucage and Carruthers, Tetra. Letts. 22:1859-1862 (1981), or the triester method (Matteucci et al., J. Am. Chem. Soc. 103:3185 (1981)), and may be performed on commercial automated oligonucleotide synthesizers. A double-stranded fragment may be obtained from the single-stranded product of chemical synthesis either by synthesizing the complementary strand and annealing the strand together under appropriate conditions, or by synthesizing the complementary strand using DNA polymerase with an appropriate primer sequence.

The natural or synthetic nucleic acid fragments coding for a desired sequence may be incorporated into vectors capable of introduction into and replication in a prokaryotic or eukaryotic cell. Usually the vectors are suitable for replication in a unicellular host, such as yeast or bacteria, but may also be introduced into cultured mammalian or plant or other eukaryotic cell lines, with or without integration within the genome. The vectors will typically comprise an expression system recognized by the host cell, including the intended recombinant nucleic acid fragment encoding the desired polypeptide. The vectors will also contain a selectable marker, i.e. a gene encoding a protein needed for the survival or growth of a host cell transformed with the vector. The presence of this gene ensures the growth of only those host cells which express the inserted nucleic acid of interest. Typical selection genes encode proteins that 1) confer resistance to antibiotics or other toxic substances, e.g. ampicillin, neomycin, methotrexate, etc.; b) complement auxotrophic deficiencies, or c) supply critical nutrients not available from complex media, e.g. the gene encoding D-alanine racemase for Bacilli. The choice of the proper selectable marker will depend on the host cell, and appropriate markers for different hosts are well known in the art. Such vectors may be prepared by means of standard recombinant techniques well known in the art (Sambrook et al., (1989); Ausubel et al., (1987)).

For gene transfer into the cells to express the selected molecules, nucleic acid may be directly introduced ex vivo in the form of “naked” nucleic acid, e.g. by microinjection, electroporation, as calcium-phosphate-DNA gels, with DEAE dextran, or in encapsulated form, e.g. in vesicles such as liposomes, or in a suitable viral vector.

Vectors containing the nucleic acid encoding the desired molecules for expression are preferably recombinant expression vectors in which high levels of gene expression may occur, and which contain appropriate regulatory sequences for transcription and translation of the inserted nucleic acid sequence. Regulatory sequences refers to those sequences normally associated (e.g. within 50 kb) of the coding region of a locus which affect the expression of the gene (including transcription of the gene, and translation, splicing, stability or the like, of the messenger RNA). A transcriptional regulatory region encompasses all the elements necessary for transcription, including the promoter sequence, enhancer sequence and transcription factor binding sites. Regulatory sequences also include, inter alia, splice sites and polyadenylation sites. An internal ribosomal entry site(IRES) sequence may be placed between recombinant coding sequences to permit expression of more than one coding sequence with a single promoter.

Exemplary transcriptional control regions include: the SV40 early promoter region, the cytomegalovirus (CMV) promoter (human CMV IE94 promoter region (Boshart et al, Cell, 41:521-530 (1985)); the promoter contained in the 3′ long terminal repeat of Rous sarcoma virus or other retroviruses; the herpes thymidine kinase promoter; the regulatory sequences of the methallothionein gene; regions from the human IL-2 gene (Fujita et al., Cell, 46:401-407 (1986)); regions from the human IFN gene (Ciccarone et al., J. Immunol. 144:725-730 (1990)); regions from the human IFN gene (Shoemaker et al., Proc. Natl. Acad. Sci. USA 87:9650-9654(1990)); regions from the human IL-4 gene (Arai et al., J. Immunol. 142:274-282 (1989)); regions from the human lymphotoxin gene (Nedwin et al., Nucl. Acids. res. 13:6361-6373 (1985)); regions from the human granulocyte-macrophage CSF gene (GM-CSF) (Miyatake et al., EMBO J. 4:2561-2568 (1985)); and others. When viral vectors are employed, recombinant coding sequences may be positioned in the vector so that their expression is regulated by regulatory sequences such as promoters naturally residing in the viral vector.

In addition, operational elements may include leader sequences, termination codons, and other sequences needed or preferred for the appropriate transcription and subsequent translation of the inserted nucleic acid sequences.

Secretion signals may also be included whether from a native protein, or from other secreted polypeptides of the same or related species, which permit the molecule to enter cell membranes, and attain a functional conformation.

It will be understood by one skilled in the art that the correct combination of expression control elements will depend on the recipient (“host”) cells chosen to express the molecules. The expression vector should contain additional elements needed for the transfer and subsequent replication of the expression vector containing the inserted nucleic acid sequences in the host cells. Examples of such elements include, but are not limited to, origins of replication and selectable markers.

The vector may contain at least one positive marker that enables the selection of cells carrying the inserted nucleic acids. The selectable molecule may be a gene which, upon introduction into the cell, expresses a dominant phenotype permitting positive selection of cells carrying the gene. Genes of this type are known in the art and include, for example, drug resistance genes such as hygromycin-B phosphotransferase (hph) which confers resistance to the antibiotic G418; the aminoglycoside phosphotransferase gene (neo or aph) from Tn5 which codes for resistance to the antibiotic G418; the dihydrofolate reductase (DHRF) gene; the adenosine deaminase gene (ADA) and the multi-drug resistance (MDR) gene.

Suitable vectors for the invention may be plasmid or viral vectors, including baculoviruses, adenoviruses, poxviruses, adenoassociated viruses (AAV), and retroviral vectors (Price et al, Proc. Natl. Acad. Sci. USA 84:156-160 (1987) such as the MMLV based replication incompetent vector pMV-7 (Kirschmeier et al., DNA 7:219-225 (1988)), as well as human and yeast artificial chromosomes (HACs and YACs). Plasmid expression vectors include plasmids including pBR322, pUC or Bluescript TM (Stratagene, San Diego, Calif.). Exemplary vectors are described e.g., in U.S. Pat. Nos. 6,040,147; 6,033,908; 6,037,172; 6,027,722; 5,741,486; 5,656,465.

Recombinant viral vectors are introduced into cells using standard infection conditions. Infection techniques have been developed which use recombinant infectious virus particles for gene delivery into cells. Viral vectors used in this way include vectors derived from simian virus 40 (SV40; Karlsson et al., Proc. Natl. Acad. Sci. USA 82:158 (1985)); adenoviruses(Karlsson etl al., EMBO J., 5:2377 (1986)); AAV (Carter, Current Opinion in Biotechnology, 3:533-539 (1992)); vaccinia virus (Moss, et. al., Vaccine, 6:161-3, 1988)); and retroviruses (Coffin, in Weiss et al., (eds.), RNA Tumor Viruses, 2nd ed. Vol. 2, Cold Spring Laboratory, New York, pp. 17-71 (1985)).

In retroviral vectors, genes are inserted so as to be under the transcriptional control of the promoter incorporated in the retroviral long terminal repeat (LTR), or by placing them under the control of a heterologous promoter inserted between the LTRs. This latter strategy provides a way of coexpressing a dominant selectable marker gene in the vector, thus permitting selection of cells that are expressing specific vector sequences.

Nonreplicating viral vectors can be produced in packaging cell lines which produce virus particles which are infectious but replication defective, rendering them useful vectors for introduction of nucleic acid into a cell lacking complementary genetic information enabling encapsidation (Mann et al., cell 33:153 (1983); Miller and Buttimore, Mol. Cell. Biol. 6:2895 (1986) (PA317, ATCC CRL9078). Packaging cell lines which contain amphotrophic packaging genes able to transduce cells of human and other species origin are preferred.

DNA vectors containing the inserted genes or coding sequences are introduced into cells using standard methods, such as electroporation, liposomal preparations, Ca-PH-DNA gels, DEAE-dextran, nucleic acid particle “guns” and other suitable methods.

In general, nucleic acid encoding the selected molecules is inserted by standard recombinant DNA methods into a vector containing appropriate transcription and translation control sequences, including initiation sequences operably linked to the gene sequence to result in expression of the recombinant genes in the recipient host. Operably linked refers to a juxtaposition wherein the components are in a relationship permitting them to function in their intended manner. For instance, a promoter is operably linked to a coding sequence if the promoter effects its transcription or expression.

The nucleic acid sequences encoding the proteins or protein fragments selected for expression in may be inserted in a single vector or in separate vectors. More than one gene encoding a selected polypeptide, or portion thereof, may be inserted into a single vector or in separate vectors.

Expression of recombinant genes of interest after introduction into APCs is confirmed by immunoassays or biological assays for functional activity of the protein product. For example, expression of introduced molecules into cells may be confirmed by detecting the binding of labeled antibodies specific for the molecules to the cells using assays well known in the art such as FACS(Fluorescent Activated Cell Sorting) or ELISA (enzyme-linked immunoabsorbent assay).

Biological activity of the engineered cells can be verified, for example, in in vitro assays. The ability of the cells of the invention to function as desired, e.g. to bind to CTLA-4 and to downmodulate an immune response or to bind to CD28 and upmodulate an immune response may be tested using standard in vitro and/or in vivo assays.

VI. Pharmaceutical Compositions

The active molecules of the invention (e.g., the constructs of the invention as well as compositions for causing an anti-CTLA-4 molecule and/or MHC molecule to be expressed on a cell) can be suspended in a any known physiologically compatible pharmaceutical carrier, such as cell culture medium, physiological saline, phosphate-buffered saline, or the like, to form a physiologically acceptable, aqueous pharmaceutical composition. Parenteral vehicles include sodium chloride solution, Ringer's dextrose, dextrose and sodium chloride, and lactated Ringer's. Other substances may be added as desired such as antimicrobials. A active molecule for donwmodulating the immune response can be incorporated into a composition, e.g., a pharmaceutical composition suitable for administration. Such compositions typically further comprise a carrier, e.g., a pharmaceutically acceptable carrier. As used herein the language “carrier” is intended to include any and all solvents, dispersion media, coatings, antibacterial and antifungal agents, isotonic and absorption delaying agents, and the like, compatible for use with cells, e.g., compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions can be included in a container, pack, or dispenser together with instructions for administration. The kit can further comprise a means for administering the active molecule of the invention, e.g., one or more syringes. The kit can come packaged with instructions for use.

VII. Uses and Methods of the Invention

A. CTLA-4

The active molecules of the invention, e.g., the constructs of the invention or the cells caused to express an antibody binding portion of anti-CTLA-4 are useful in downmodulating the immune response. The present invention provides for both prophylactic and therapeutic methods of treating a subject at risk of (or susceptible to) a disorder or having a disorder associated with an aberrant or undesirable immune response.

The active molecules of the invention can be used to downmodualte both primary and secondary immune responses. They can be used to downmodulate immune responses mediated, either directly or indirectly (e.g., based on helper function) by T cells. In one embodiment, the subject compositions and methods are used to downmodulate CD4+ T cell responses. In another embodiment, the subject compositions and methods are used to downmodulate CD8+ T cell responses.

In one aspect, the invention provides a method for preventing an undesirable immune response in a subject. Administration of an active molecule of the invention can occur prior to the manifestation of symptoms for which modulation of the immune response would be beneficial, such that a disease or disorder is prevented or, alternatively, delayed in its progression. Such administration can be used to prevent or downmodulate primary immune responses. Another aspect of the invention pertains to methods of modulating an immune response for therapeutic purposes.

The present invention provides methods of treating an individual afflicted with a disease or disorder that would benefit from downmodulation of the immune response using the constructs of the invention or by causing a cell to express an antibody binding portion of an anti-CTLA-4 antibody. The constructs of the invention can be administered ex vivo (e.g., by contacting the cell with the agent in vitro) or, alternatively, in vivo (e.g., by administering the construct to a subject). Likewise, a cell can be made to express an antibody binding portion of an anti-CTLA-4 antibody either in vivo or ex vivo.

The instant compositions and methods can be used to downmodulate immune responses to endogenous peptides or exogenous peptides. In one embodiment of the invention, the immune response against a specific antigen(s) is downmodulated by co-administering an antigen with an active molecule of the invention. In another embodiment, MHC molecules are loaded with an antigen against which the immune response to be downmodulated is directed. In one embodiment, by so doing, downregulation of an immune response to a specific proteins (e.g., therapeutic proteins, transplantation antigens, allergans, self antigens, etc.)

Downmodulation of the immune response is useful to downmodulate the immune response, e.g., in situations of tissue, skin and organ transplantation, in graft-versus-host disease (GVHD), or in autoimmune diseases such as systemic lupus erythematosus, and multiple sclerosis.

For example, blockage of immune cell function results in reduced tissue destruction in tissue transplantation. Typically, in tissue transplants, rejection of the transplant is initiated through its recognition as foreign by immune cells, followed by an immune reaction that destroys the transplant. The administration of an active molecule of the invention prior to or at the time of transplantation, can inhibit the immune response. In one embodiment, a cell for transplantation is caused to express an antibody binding portion of an antibody that binds CTLA-4.

In one embodiment, use of the active molecules of the invention is sufficient to anergize the immune cells, thereby inducing tolerance in a subject. In one embodiment, long term tolerance is induced in a subject and may avoid the necessity of repeated administration of these blocking reagents.

To achieve sufficient immunosuppression or tolerance in a subject, it may also be desirable to block the costimulatory function of other molecules such as CTLA-4. For example, it may be desirable to block the function of B7-1, B7-2, or B7-1 and B7-2 by administering a soluble form of a combination of peptides having an activity of each of these antigens or blocking antibodies against these antigens (separately or together in a single composition). Other downmodulatory agents that can be used in connection with the downmodulatory methods of the invention include, for example, soluble forms of CTLA-4, blocking antibodies against other immune cell markers or soluble forms of other receptor ligand pairs (e.g., agents that disrupt the interaction between CD40 and CD40 ligand (e.g., anti CD40 ligand antibodies)), antibodies against cytokines, fusion proteins (e.g., CTLA-4-Fc), and immunosuppressive drugs, (e.g., rapamycin, cyclosporine A or FK506).

The active molecules of the invention are also useful in treating autoimmune disease. Many autoimmune disorders are the result of inappropriate activation of immune cells that are reactive against self tissue and which promote the production of cytokines and autoantibodies involved in the pathology of the diseases. Preventing the activation of autoreactive immune cells may reduce or eliminate disease symptoms. The active molecules of the invention are useful to inhibit immune cell activation and prevent production of autoantibodies or cytokines which may be involved in the disease process.

Inhibition of immune cell activation can also be used therapeutically in the treatment of allergy and allergic reactions, e.g., by inhibiting IgE production. An active molecule of the invention can be administered to an allergic subject to inhibit immune cell mediated allergic responses in the subject. Administration of an active compound can be accompanied by exposure to allergen. Allergic reactions can be systemic or local in nature, depending on the route of entry of the allergen and the pattern of deposition of IgE on mast cells or basophils. Thus, inhibition of immune cell mediated allergic responses can be effected locally or systemically by administration of an active molecule of the invention.

B. CD28

The active molecules of the invention, e.g., the constructs of the invention or the cells caused to express an antibody binding portion of anti-CD28 are useful in enhancing the immune response. The present invention provides for both prophylactic and therapeutic methods of treating a subject at risk of (or susceptible to) a disorder or having a disorder associated with an aberrant, absent or undesirable immune response.

The active molecules of the invention can be used to enhance both primary and secondary immune responses. They can be used to enhance immune responses mediated, either directly or indirectly (e.g., based on helper function) by T cells. In one embodiment, the subject compositions and methods are used to enhance CD4+ T cell responses. In another embodiment, the subject compositions and methods are used to enhance CD8+ T cell responses.

In one aspect, the invention provides a method for enhancing a desirable immune response in a subject. Administration of an active molecule of the invention can occur prior to the manifestation of symptoms for which modulation of the immune response would be beneficial, such that a disease or disorder is prevented or, alternatively, delayed in its progression. Such administration can be used to induce or enhance primary immune responses. Another aspect of the invention pertains to methods of modulating an immune response for therapeutic purposes.

The present invention provides methods of treating an individual afflicted with a disease or disorder that would benefit from enhancement of the immune response using the constructs of the invention or by causing a cell to express an antibody binding portion of an anti-CD28 antibody. The constructs of the invention can be administered ex vivo (e.g., by contacting the cell with the agent in vitro) or, alternatively, in vivo (e.g., by administering the construct to a subject). Likewise, a cell can be made to express an antibody binding portion of an anti-CD28 antibody either in vivo or ex vivo. The instant compositions and methods can be used to enhance immune responses to endogenous peptides or exogenous peptides. In one embodiment of the invention, the immune response against a specific antigen(s) is enhanced by co-administering an antigen with an active molecule of the invention. In another embodiment, MHC molecules are loaded with an antigen against which the immune response to be enhanced is directed. In one embodiment, by so doing, enhancement or induction of an immune response to a specific proteins (e.g., therapeutic proteins, tumor antigens, viruses, and the like).

Induction or enhancement of the immune response is useful to upregulate the immune response, e.g., in situations of tumor vaccination, in viral immunity, or in immunodeficiency diseases such as AIDS, and DiGeorges Syndrome.

For example, induction/enhancement of immune cell function results in increased tumor destruction in cancer patients. Typically, in cancer patients, progression of tumor growth can be prevented through its recognition as foreign by immune cells, followed by an immune reaction that destroys the tumor. The administration of an active molecule of the invention prior to or at the time of tumor detection, can inhibit the immune response. In one embodiment, a tumor cell is caused to express an antibody binding portion of an antibody that binds CD28.

In one embodiment, use of the active molecules of the invention is sufficient to activate the immune cells, thereby inducing active immunity in a subject. In one embodiment, long term immunity is induced in a subject and may avoid the necessity of repeated administration of these augmenting reagents.

To achieve sufficient immune activation in a subject, it may also be desirable to block the downregulatory function of other molecules such as CTLA-4. For example, it may be desirable to block the function of the inhibitory receptor for B7-1 and B7-2 by administering agents that compete with the binding of the inhibitory receptor for their natural ligands, but that fail to transmit an inhibitory signal (e.g., soluble forms of these polypeptides or blocking antibodies). Other activating agents that can be used in connection with the activating methods of the invention include, for example, activating antibodies against other immune cell markers or soluble forms of other receptor ligand pairs (e.g., agents that enhance the interaction between CD40 and CD40 ligand (e.g., anti CD40 ligand antibodies)), adjuvants, cytokines such as IL-2 or IL-15, and superantigens.

The active molecules of the invention are also useful in treating infectious diseases. Many viral diseases are the result of inefficient activation of immune cells that are needed to destroy the infectious agents and which produce cytokines and antibodies involved in the irradication of the infectious agent. Enhancing the activation of reactive immune cells may reduce or eliminate disease symptoms. The active molecules of the invention are useful to enhance immune cell activation and increase production of antibodies or cytokines which may be useful in the disease process.

Activation of immune cell expansion can also be used therapeutically in the treatment of AIDS and Immunodeficiency disease by increasing T cell numbers and function. An active molecule of the invention can be administered to an AIDs subject to enhance immune cell expansion in the subject. Administration of an active compound can be accompanied by exposure to viral antigen.

VIII. Administration of Constructs and/or Molecules of the Invention

The active molecules may be introduced into the subject to be treated by using one of a number of methods of administration of therapeutics known in the art. For example, active molecules may be inoculated (with or without adjuvant) parenterally (including, for example, intravenous, intraperitoneal, intramuscular, intradermal, and subcutaneous), by ingestion, or applied to mucosal surfaces. Alternatively, the active molecules of the invention are administered locally by direct injection into a cancerous lesion or infected tissue. “Inoculation” refers to administration of the active molecules of the invention to a subject.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition will be sterile and should be fluid to the extent that easy syringability exists. A composition will be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Active molecules of the invention can be introduced into a subject with an antigen or antigens corresponding to those to which an immune response to be downmodulated is directed. Such molecules can be introduced into a subject prior to onset of an immune response or when an immune response is ongoing.

A “therapeutically effective amount” of a composition of the invention is a dose sufficient to reduce or suppress an immune response to the selected antigen.

Routes of administration include epidermal administration including subcutaneous or intradermal injections. Transdermal transmission including iontophoresis may be used, for example “patches” that deliver product continuously over periods of time.

Mucosal administration of the active molecules of the invention is also contemplated, including intranasal administration with inhalation of aerosol suspensions. Suppositories and topical preparations may also be used. The methods of the invention contemplate the dosage of a sufficient amount or number of the active molecules to downmodulate T response(s) in a subject. The active molecules may be introduced in at least one dose and either in that one dose or through cumulative doses are effective in reducing an immune response. The active molecules are administered in a single infusion or in multiple, sequential infusions.

Different subjects are expected to vary in responsiveness to such treatment. Dosages will vary depending on such factors as the individual's age, weight, height, sex, general medical condition, previous medical history, and immune status. Therefore, the amount or number of active molecules infused as well as the number and timing of subsequent infusions, is determined by a medical professional carrying out the therapy based on the response of the patient.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

After administration, the efficacy of the therapy can be assessed by a number of methods, such as assays that measure T cell proliferation, T cell cytotoxicity, antibody production, and/or clinical response. An decrease in the production of antibodies or immune cells recognizing the selected antigen will indicate a downmodulated immune response. Efficacy may also be indicated by improvement in or resolution of the disease (pathologic effects), associated with the reduction or disappearance of the unwanted immune response, or improvement in or resolution of the disease (pathologic effects) associated with the unwanted immune response (e.g. autoimmune disease) allergic reaction or transplant rejection). For example, standard methodologies can be used to assay, e.g., T cell proliferation, cytokine production, numbers of activated T cells, antibody production, or delayed type hypersensitivity. In addition or alternatively, improvement in a specific condition for which treatment is being given can be monitored.

The practice of the present invention employs conventional techniques of molecular biology, microbiology, recombinant DNA, and immunology, within the skill of these arts. Such techniques are found in the scientific literature (See, e.g., Brock, Biology of Microorganisms, Eighth Ed., (1997), (Madigan et al., eds.), Prentice Hall, Upper Saddle River, N.J.; Sambrook et al., Molecular Cloning: A Laboratory Manual, Second Ed., (1989); Oligonucleotide Synthesis, M. J. Gait Ed., 1984, Animal Cell Culture, Freshney, ed., 1987; Methods in Enzymology, series, Academic Press, Inc.; Gene Transfer Vectors for Mammalian Cells, Miller and Calos, Eds., 1987; Handbook of Experimental Immunology, Weir and Blackwell, Eds., Current Protocols in Molecular Biology. Ausubel et al, Eds., 1987, and Current Protocols in Immunology, Coligan et al., Eds., 1991). These references are incorporated in their entirety herein by reference.

This invention is further illustrated by the following examples which should not be construed as limiting. The contents of all references, patents and published patent applications cited throughout this application, as well as the Figures and the Sequence Listing are incorporated herein by reference.

### EXAMPLES

The following materials and methods were used throughout the Examples:

Experimental Animals and Cell Lines: Female mice between 6 and 12 weeks of age were used for all experiments. Mice were maintained in the University of Chicago animal housing facility in a specific pathogen-free environment. BALB/c mice were purchased form Frederick Cancer Research and Development Center (National Cancer Institute, Frederick, Md.). DO11.10 transgenic mice (carrying a class II restricted transgenic TCR specific for OVA) and 2C transgenic mice (carrying a class I restricted transgenic TCR specific for a self peptide) are known in the art (see, e.g., Oosterwegel et al. 1999. J. Immunol. 163:2634; Kuhns et al. 2000 Proc. Natl. Acad. 97:756; Cook et al. 1997. Immunity 7:233) and were also used in these Examples. Human embryonic kidney cell line 293 are also known in the art and were used in the instant Examples. The murine B cell Lymphoma cell line was purchased from the American Type Culture collection. All cell cultures were carried out at 3TRC. 10% CO₂ in Dulbecco Modified Eagle's Minimum Medium (DMEM) (Life Technologies LTD. Grand Island N.Y.) supplemented with 5% or 10% Fetal Calf Serum. 25 μm HEPES (Life Tchnologies). 2 mM L-glutamine (Life Technologies). 100U/ml penicillin (Sigma-Aldrich), St. Louis, Mo.). 100 μg/ml streptomycin (Sigma Aldrich) 2 mM non-essential amino acids (Life Technologies). And 5 μM 2-mercaptoethanol (Sigma-Aldrich). Adherent cell lines were lifted by incubation with a 0.02% solution of Na₂EDTA (Sigma-Aldrich) in Phosphate Buffered Saline (PBS). All tissue culture ware was purchased from Becton Dickinson (Franklin Lakes, N.J.).

Generation ofSurface-Linked scFv and 1-A^{d} expression constructs The generation and functional characteristics of surfaced-linked scFv constructs derived from the hamster mAbs 145-2C11 (anti-murineCD3ε; Leo 0 et al., Proc Natl Acad Sci U S A. 1987. 84: 1374-8) and PV-1 (anti-murine CD28) and 5H7 (anti-humanClassl MHC; Smith D, et al. J Immunol 1994. 153: 1054) are known in the art. Surface-expression for these proteins has been achieved using glycosylphosphatidylinositol (GPI) anchor motif. An anti-CTLA-4 scFv construct (4F10scFv) was generated from the hybridoma UC-10-4F10 which secretes a mAb with specific binding affinity for murine CTLA-4. Total RNA prepared from hybridoma cells by the guanidinium isoiniocyanate CsCL method was used to synthesize cDNA with the First Strand cDNA kit (Novagen, Madison, Wis.) according to the manufacturer's instructions. Amplification of the VH gene from cDNA was preformed with the following primers: Sense: 5′ CGAATGATGCATCC(C/G)AGGTG(CA)AGCTG(C/G/A)(A/T)G(G/C)AGTC 3′ which incorporates an Nsil restriction site (in bold): Antisense: 5′ GCAAATAAGCTTTTGTTCGGCTGAGGAGACGGT(G/A)AC 3′ which incorporates a HindIII restriction site (in bold). Amplification of the V_{L} gene was performed with the primers: Sense 5′ CGAATGGACGTCATGATGACACAGTCTCC 3′ which incorporates an AatII restriction site (in bold): Antisense: 5′ TATGATCCGCGGAGGAACGTTT(T/G)ATTTCCAGCTTGGTCCC 3′ which incorporates a SacII restriction site (in bold). Cycling conditions were (93° C.×1 minute. 50° C×1 minute. 72° C.×1 minute for 35 cycles using the DNA polymerase Pfu (Stratagene, La Jolla, Calif.). The PCR products of the V_{L} and V_{H} genes were digested with the restriction endonucleases Aat II/SacII and Nsi I/Hind III respectively. The digested PCR products were cloned into the temperature-inducible expression vector Genex in the orientation NH₂-4F10 VL-linker-4F10 VH-COOH. Surface-linked 4F10scFv (subsequently referred to as mem4F 10scFv) was constructed by a tailed primer Polymerase Chain Reaction (PCR) strategy using 4F10scFv as a template. The primers used were: Sense: 5′ GAGTAAGCTTATGAGGACCCCTGCTCAGTTTCTTGGAATCTTGTTGCTCTGGTTT CCAGGTATCAAATGTGACGTCATGATGACACAGTCTCC 3′ which incorporates HindIll restriction site (in bold). A murine light chain leader peptide sequence (underlined) and nucleotide residues 4 to 26 of the sequence for 4F10scFv (in italics). Antisense: 5′ AGCTTCTTAAGCTTCCGCTACCACTAGACACAGGGGCCAGTGGATAGACCGATG GGGCTGTTGTTTTGGCGGCTGAGGAGACGGTGACC 3′ which incorporates an AfIII restriction site (in bold) sequence encoding a flexible spacer peptide (underlined) and the final 19 residues of the sequence for 4F10scFv. Cycling conditions used to generate the modified 4F10scFv with these primers were: (94° C.×1 minute). 1 cycle (94°×30 seconds. 58°×30 seconds. 72° C.×1 minute). 5 cycles: (94°×30 seconds 72° C.×1 minute). 30 cycles: (72° C.×10 minutes). 1 cycle. Amplification was carried out on a Geneamp 9600 thermal cycler (Perkins Elmer Corp., Norwalk. CT) using Taq DNA polymerase (Life Technologies). This resulted in a single product which was then digested with the restriction endonucleases HindIII and AfIII (Life Technologies). Two surface-linkage strategies were used to generate a membrane bound form of the modified 4F10scFv (mem-4F10scFv). The first utilized a GPI anchor motif while the second utilized a cDNA fragment corresponding to the entire transmembrane domain and the first 34 amino acid residues of the cytoplasmic domain of the murine B7-1 (CD86) protein. The latter was generated by PCR using cDNA encoding murine B7-1 (CD80) as a template and with the following primers: Sense: 5′ GAGCTGCTTAAGCAAGAACACACTTGTGCTC 3′ which includes an AfIII site (in bold) and the first 20 nucleotides of the B7-1 transmembrane domain)in italics). Antisense: 5′ GTTCGCTCTAGACTAAAGGAAGACGGTCTGTTCAGC 3′ which incorporates and Xbai site (in bold) and in-frame stop codon (underlined) and 22 nucleotide residues from the intracytoplasmic domain of B7-1 (in italics). Cycling conditions for generation of the surface-linkage domain were: (94° C.×1 minute), 1 cycle (94° C.×30 seconds, 57° C.×30 seconds, 72° C.×1 minute), 35 cycles: (72° C.×10 minutes), 1 cycle. The modified 4F10scFv product was digested with the restriction endonucleases HindIII/AfIII while the surface-linkage motifs were digested with the enzyme pair AfIII/Xbal. Final constructs were then assembled by simultaneous ligation of the digested 4F10scFv and the digested surface-linkage motifs into the HindII and Xbal sites of the mammalian expression vector pCDBA3.1 (+) (Invitrogen Corp. Carlisbad, Calif.) at the HindIII and Xbal sites to generate mem4F10scFvpCDNA3.1(+). Complementary DNA sequences for the α and β chains of the murine Class II MHC protein I-A^{d} were generated by reverse transcription and polymerase chain reaction (RT-PCR). Total RNA was extracted from the BALB/c-derived B cell lymphoma cell line A20 using Trizol Reagent (Life Technologies) according to the manufacturers instruction. Reverse transcription with an oligo-dT primer was carried out using the Superscript II first strand cDNA synthesis kit (Life Technologies) by recommended protocol and was followed by PCR using the DNA polymerase mixture Elongase (Life Technologies). Primer for PCR amplification of the coding region of the two chains were derived from published sequence and were as follows: I-A^{d}α: Sense—5′ GAGCTGAAGCTTATGCCGTGCAGAGCTCTGATTC TGG 3′ (HindIII site in bold). Antisense—5′ GCCCGCTCTAGATCATAAAGGCCCTGGGTGTCTGG 3′ (Xbal site in bold): I-A^{d}β Sense—5′ GAGCTGAAGCTTATGGCTCTGCAGATCCCCAGC 3′ Antisense—5′ GCCCGCTCTAGATCACTGCAGGAGCCCTGCTGGAGG 3′. Conditions for PCR were: (94° C.×1 minute) 1 cycle: (94° C.×1 minute) 1 cycle: (94° C.×30 seconds, 57° C.×30 seconds, 68° C.×30 seconds) 35 cycles: (68° C.×10 minutes) 1 cycle. The resulting products were digested with the restriction enzymes HindIII and Xbal and ligated into the expression vectors pCDNA3.1 (+)(I-A^{d}α) and pCDNA3.1 (+)ZEO(I-A^{d}β).

Transient Transfection of 293 cells: Cells were transiently transfected with one or more plasmid constructs by calcium phosphate precipitation. The constructs and amounts of DNA used in individual experiments are indicated in the relevant figure legends. Subconfluent cells were plated in 10 cm tissue culture dishes at 2×10⁶ cells per dish. Two hours later precipitates were prepared by mixing plasmid DNA in 500 μl of 0.24MCaC12 and 500 μl of Hepes Buffered Saline (300 mM NaCl₂ 1.5 mM Na₂HPO₂7H₂O. 50Mm Hepes) with agitation. Precipitates were then added dropwise to adherent 293 cells and removed 18 hours later by exchange of medium. Transfectants were lifted for flow cytometric analysis and for use in co-culture experiments between 36 and 48 hours after transfection.

Flow Cytometric Analysis: Transiently transfected 293 cells were lifted, washed and resuspended in FACs Buffer (PBS. 0.1% BSA. 0.01% NaCl₂. Aliquots of 100 μl were incubated with and without Fluorescein-coupled soluble murine fusion proteins (mCTLA-4Ig or mCD28Ig) for 30 minutes at 4° C., washed, resuspended in 250 μl of FACs buffer and analyzed on a Facscan™ Flow Cytometer (Becton Dickinson) with the CellQuest™ software package. The soluble fusion proteins murine CD28 μg and murine CTLA-4 μg used for flow cytometric analysis were provided by Genetics Institute, Cambridge, Mass.

Purification and Pre-activation ofMurine Lymph Node T Cells: Purified T cells were prepared by dissection of inguinal, axillary and mesentric lymph nodes followed by gentle disruption between two sterile frosted glass slides and re-suspension in complete medium. Cell suspensions were then incubated in a nylon wool column for 1 hour at 37° C. and non-adherent cells eluted in 30 ml of sterile PBS with 5% PCS. Eluted cells were incubated with hybridoma supernatants containing mAbs against heat stable antigen (hybridoma JIId) and class II MHC (hybridoma MKD6) for 30 minutes at 4° C. For purification of CD4⁺ T cell or CD8⁻ T cell populations supernatants containing mAbs against CD4 (hybridoma RL172.4) or against CD8 (hybridoma 3.155) were also added. Antibody binding cells were depleted by addition of an equal volume of rabbit complement (Pel Freez Clinical Systems, Brown Deet, Wis.) diluted 1.5 in sterile PBS with incubation at 37° C. for 45 minutes. Viable cells were then isolated by density-gradient centrifugation using Ficoll-Hypaque. Purity of the desired cell populations was between 95% and 99%. For studies in which pre-activated T cells were employed freshly purified T cells were added to six-well tissue culture plates which had been coated with goat anti-hamster IgG (10 μg/ml in PBS. Cappel.) followed by 145-2C11 and PV-1mAb(2 μg/ml each in PBS). Between 60 and 72 hours later the cells were removed, washed in complete medium, and cultured at 37° C. for a further 8 hours prior to use in co-culture studies.

In vitro Proliferation and Cytokine Assays: Transiently transfected 293 cells were treated with Mitomycin C (Sigma-Aldrich) 50 μg/ml for 1 hour at 37° C. followed by extensive washing then added to wells of 96-well, flat bottomed tissue culture plates. The numbers of cells used per well for individual experiments are indicated in the relevant figure legends. In experiments where mixtures of 293 transfectants were used, cell suspensions were pre-mixed at 1:1 ratio just prior to their addition to the wells. Resting or preactivated murine lymph node T cells were then added to wells containing transfectants and incubated at 37° C. At defined time-points plates were pulsed with 1 μCi of tritiated thymidine and incubated for a further 8 to 16 hours. Plates were freeze-thawed and then harvested onto fiberglass filters and analyzed using a filtermate 196 cell harvester and Topcount Scintillation Counter (Packard Instrument Co., Meridian, Conn.) Three or six identical wells were analyzed for each condition and results (in cpm) expressed as mean±SD. In all cases background thymidine incorporation for Mytomycin C-treated transfectants was determined and was subtracted from final counts for experimental conditions. For analysis of Cytokine concentrations culture supernatants were withdrawn from wells at defined time-points and levels of the cytokines IL-2, IL-4 and IFNy measured by commercial ELISA kits according to the manufacturer's recommendations (Endogen, Inc. Woburn, MA-IL-2 and IL-4: Pharmingen, SanDiego, CA-IFNγ).

Detection of T Cell Protein Tyrosine Phosphorylation: Aliquots of 5×10⁶ pre-activated lymph node T cells were mixed on ice in 1.5 ml tubes with aliquots of 2.5×10⁶ transiently transfected 293 cells suspended in complete culture medium. The transfectants used in individual experiments are indicated in the relevant figure legends. Cell mixtures were then pelleted by brief configuration, transferred to a heating block pre-warmed to 37° C. and incubated for 2-5 minutes followed by Lysis in 1% nonidet P-40. 50 mM tris-HCI (pH 7.4). 150 mM NaCl. 20 mM EDTA (pH 8.0). 1 mM sodium vanadate, leupeptin (110 g/ml, 10 μm aprotinin. LmM phenylsulfonylsulfoxide Lysates were precleared once with protein A sepharose beads (Amersham Pharmacia Biotech. Piscataway, N.J.) and once with protein A sepharose beads coated with an irrelevant hamster mAb (UC3-10A6). Immunoprecipitation was performed overnight at 4° C. with the anti-phosphotyrosine (pTyr) mAb FB2 coated onto Protein A sepharose beads. Immunoprecipitates were separated onto a reducing 12% SDS-PAGE gel and transferred to a polyvinylidene difluoride membrane (Millipore, Bedford, Mass.) and immunoblotted with mAb 4G10 to pTyr (Upstate Biotechnology Inc., Lake Placid, N.Y.). Bound proteins were detected by enhanced chemiluminescence according to the manufacturer's instructions (Pierce, Rockford, Ill.).

### Example 1

### Modified Anti-CTLA-4 and Anti-CD28scFvs are Expressed at the Surface of Eukaryotic Cells and Bind Appropriate Soluble Ligands

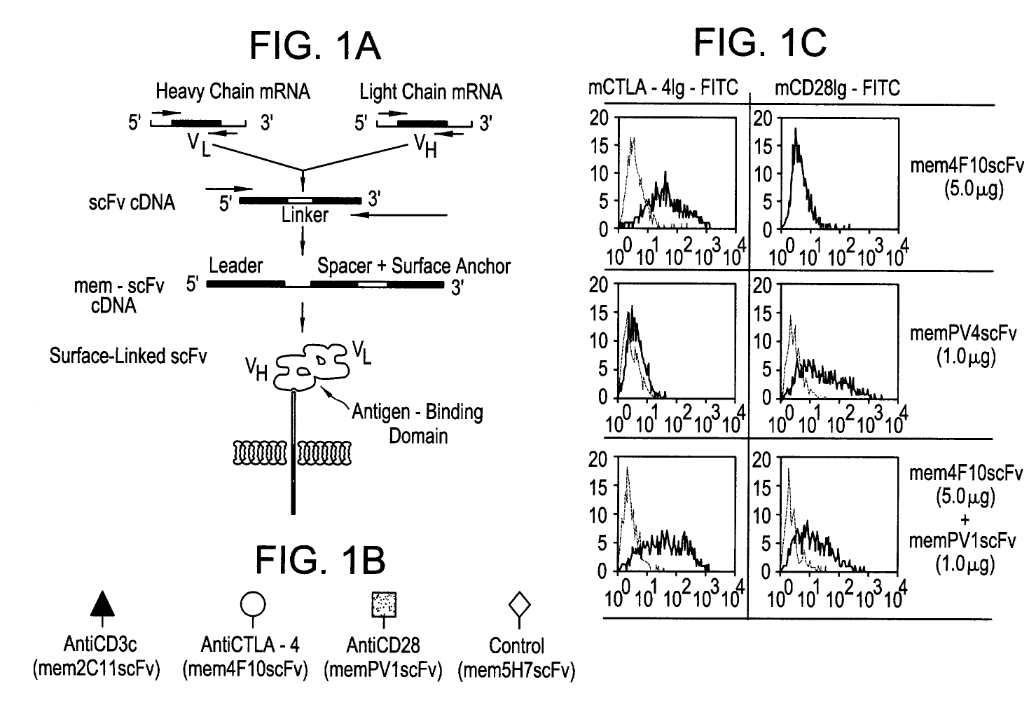

The construction strategy for the surface-linked single chain antibodies used for these studies is outlined in diagrammatic form in FIG. 1A. Panel A of FIG. 1 illustrates that sequences encoding the variable regions of the light and heavy chains of a monoclonal antibody (V_{L} and V_{H}) were amplified from RNA by RT-PCR using primers derived from constant regions and joined by sequence for a flexible peptide linker. This basic scFv cDNA is further modified by a second round of PCR using primers with extended tails to add a murine light chain 5′ leader peptide and a 3′ flexible spacer and surface linkage (anchor) motif. The modified constructs (mem-scFvs) were ligated into a mammalian expression vector for subsequent transient and stable transfections of eukaryotic cell lines. When transfected into eukaryotic cell lines, results in surface expression of a protein in which the V_{L} and V_{H} domains reconstitute the antigen-binding domain of the parent mAb. Panel B shows four surface-linked scFvs generated by the strategy outlined in panel A were used for the creation of artificial APC populations. The symbols shown were employed in subsequent figures to represent the expression characteristics of the stimulating cells used in individual experiment. Panel C shows Human embryonic kidney (293) cells, transiently transfected with CDNA encoding anti-CTLA-4 (mem4F 10), anti-CD28 (memPVI) scFv. In the amounts shown or with both constructs together were incubated with (solid lines) or without (dashed lines) FITC-coupled soluble murine fusion proteins mCTLA-4Ig and mCD28Ig as indicated and analyzed by flow cytometry for surface staining.

Transfection with mem4F10scFv (upper panels) results in surface staining by mCTLA-4Ig-FITC but not mCD28-FITC while transfection with memPVIscFv (middle panels) produces the opposite staining pattern. Doubly transfected cells are capable of binding both labeled fusion proteins (lower panels).

Three mem-scFvs were derived in this way from the mAbs 145-2C11 (hamster anti-murineCD3ε). PV-1 (hamster anti-murineCD28), and UC10-4F10 (hamster anti-murine CTLA-4). A fourth mem-scFv, derived from the mAb 5H7 (mouse anti-human Class I MHC) was also used. As this antibody has no cross-reactivity with murine antigens, this construct was employed as a control protein. Surface-linkage was mediated by a GPI anchor motif for mem2C11scFv, memPVIscFv and mem5H7scFv. For mem4F10scFv two surface-linkage strategies (a GPI anchor and a portion of the murine B7-1 protein) were developed and tested. While similar results were achieved with both of these constructs high levels of surface expression were more consistently achieved using the B7-1 derived motif and it is this protein which was utilized in the experiments reported below. Surface expression and ligand binding of mem4F10scFv and memPVIscFv were confirmed by flow cytometric analysis of human embryonic kidney (293) cells transiently transfected with one or both of these two cDNA constructs. As shown in FIG. 1C, cells expressly mem4F10scFv bound fluorchrome-labeled soluble murine CTLA-4 (mCTLA-4Ig) but not soluble murine CD28 (mCD28Ig) while those expressing memPVIscFv bound mCD28Ig but not mCTLA-4Ig. Co-transfection of both constructs resulted in binding of both soluble fusion proteins at levels comparable to those in singly-transfected cells. Thus, unlike B7-expressing cells, transfected cells could to used to specifically engage CTLA-4 either in the presence or absence of CD28 engagement. The ability of mem2C11scFv and memPVIscFv to activate murine T cells when expressed on the surface of eukaryotic cells was demonstrated in co-incubation studies of transiently transfected 293 cells and resting T cells. Untransfected 293 cells induced no proliferation of whole murine lymph node cells or purified T cells ruling out the possibility of a productive response to xenogeneic antigens and unidentified co-stimulatory ligands on these cells. Proliferation of purified T cells was induced by the expression of mem2C11 scFv on 293 cells. This proliferation was greatly enhanced by co-expression of the membrane-bound anti-CD28scFv either on the same cell surface (co-stimulation “in cis”) or, to a lesser degree, on that of a second population of transfectants (“in trans”). The level of expression induced by combined transfection with low amounts of cDNAs for mem2C 11 scFv and memPVIscFv (0.5 to 1.0 μg of each construct per 2×10⁶ cells) is sufficient to induce a strong proliferative response in resting T cells. Together these results show that the memscFv constructs are expressed, as predicted, at the cell surface, specifically bind appropriate soluble ligands, and transmit signals to murine T cells through cell-cell interactions. Thus, the capacity to rapidly and simultaneously express a number of constructs on a single population of cells allows a wide range of experimental conditions to be tested efficiently.

### Example 2

### Surface-Linked Anti-CTLA-4scFv Reduces Proliferation and IL-2 Secretion of T Cells in Primary Stimulation Cultures

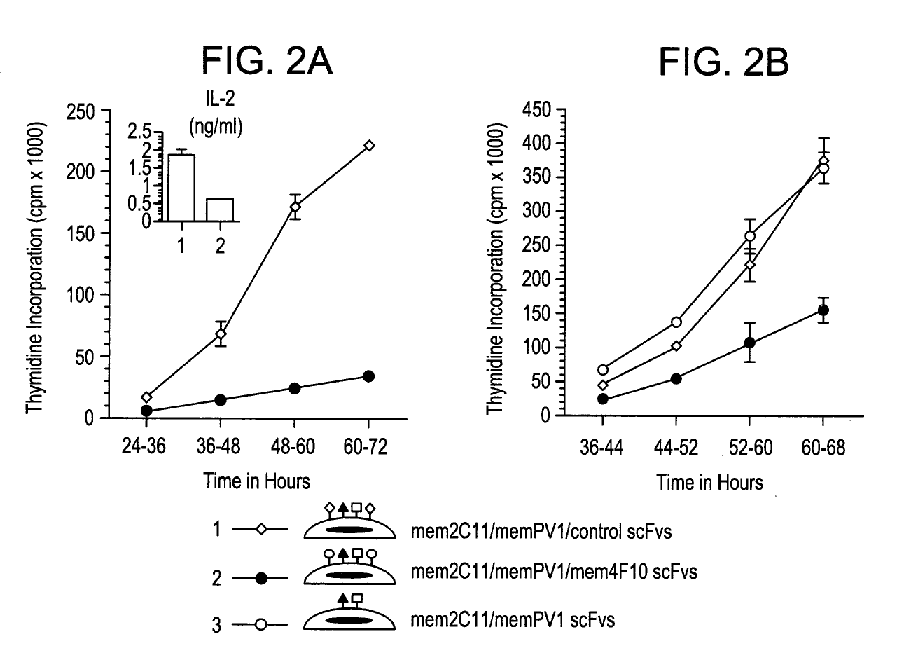

The functional properties of mem-4F10scFv were first characterized in a series of experiments in which 293 cells, transfected with combinations of the surface-linked scFvs, were co-incubated with resting murine T cells. FIG. 2 shows the results of two experiments in which 293 cells transfectants expressing mem2C11scFv and memPVIscFv were used to stimulate purified resting T cells. The effects of co-transfection with either control scFv or mem-4F10scFv were compared (FIG. 2A). Panel A of FIG. 2 shows 293 cells transiently transfected with low levels of mem2C11scFv (0.5 μg) and memPVIscFv (1.0 μg) along with higher levels (5.0 μg) of either control scFv, or mem4F10scFv. Following mitomycin C treatment transfectants were co-incubated with purified resting murine T ells. Proliferation was assessed by thymidine incorporation with pulses carried out at twelve hour intervals during the second and third day of co-culture. The combination of anti-CD3ε and anti-CD28 scFvs with control scFv induced a strong proliferative response on resting T cells. Co-expression of anti-CTLA-4scFv results in significantly lower T cell proliferation and IL-2 concentration in culture supernatants after 48 hours of co-incubation. Panel B shows proliferative responses induced by 293 cells transfected with mem2C11scFv (0.5 μg) and memPVIscFv were comparable following co-transfection with 5.0 μg of either empty vector or control scFv while co-transfection with 5.0 μg of mem4F10scFv resulted in significant attenuation. Results are expressed as the mean±SD of six identical wells for each condition.

Co-expression of the anti-CD3ε, anti-CD28 and control scFvs induced a strong proliferative response. In contrast, co-expression of antiCLTA-4scFv was associated with substantially reduced T cell proliferation and reduced IL-2 secretion. A similar result was observed in a repeated experiment (FIG. 2B). In addition, the proliferative response to combined CD3 and CD28 engagement was found to be comparable in the presence or absence of control scFv. In subsequent experiments therefore, cells transfected with equivalent amounts of empty vector or mem-4F10scFv were used. The results demonstrate that selective engagement of CTLA-4 by the engineered surface-linked ligand results in a significant attenuation of both proliferation and IL-2 production in resting T cells receiving combined TCR and CD28 signals. Furthermore they confirm that expression of mem-scFvs on a cell surface can mediate functional effects during cell-cell interactions which faithfully recreate the effects of the parent mAbs following their cross-linkage or presentation on Fc receptor.

### Example 3

### Engagement of CTLA-4 by Surface-Linked Anti-CTLA-4scFv During Secondary Stimulation of T Cells and T Cell Subsets Attenuates Proliferation and Cytokine Secretion

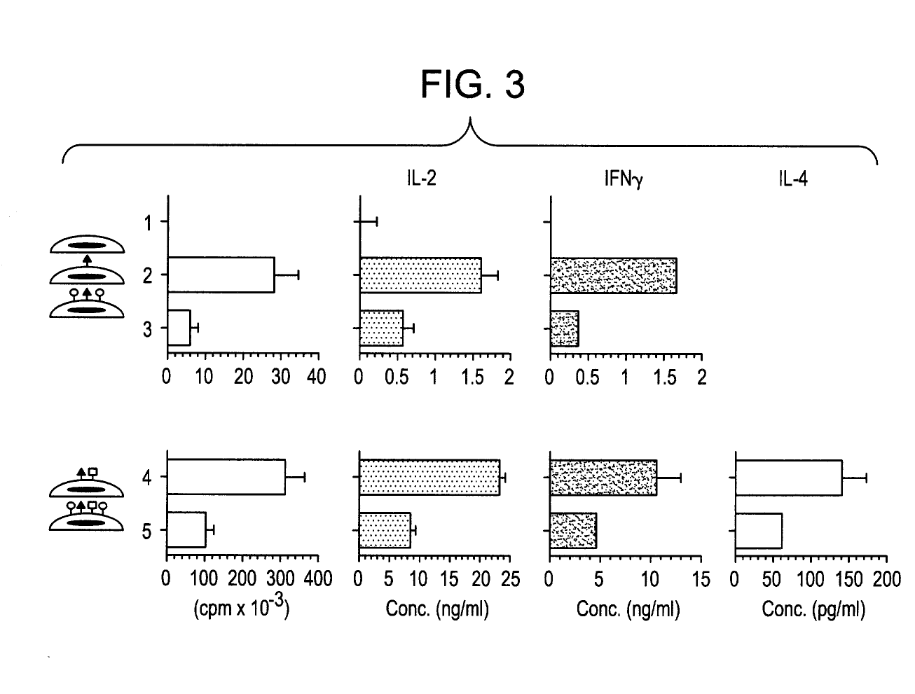

While the initial characterization of CTLA-4 mediated negative regulation were performed in primary stimulation cultures of resting T cells more recent reports have highlighted its potential role in controlling the expansion and effector function of both CD4+ and CD8+activated T cells. The concept of an extended role for CTLA-4 in shaping the magnitude nature and duration of an ongoing immune response is very much compatible with its persistent expression in activated helper and cytotoxic T cells, in memory T cells, and in T cell-derived clones of a variety of phenotypes. With this in mind we next performed experiments using T cells and T cell subsets which had been pre-activated by a combination of plate-bound anti-CD3_{ε}, and antiCD28 parent mAbs. Following this primary stimulation T cells express significant amounts of CTLA-4 predominantly in intracellular compartments, and can be subsequently induced to rapidly proliferate and secrete a variety of cytokines upon re-stimulation by a TCR signal alone or in combination with a CD28 signal. FIG. 3 shows the results of such a re-stimulation assay. Artificial APCs expressing low levels of mem2C11 scFv alone (upper graphs) or a combination of mem2C11scFv and memPVIscFv (lower graphs) were co-transfected with either empty vector or mem4F10scFv and cultured with pre-activated T cells. Peak proliferative responses and levels of IL-2, IFNγ and IL-4 are shown. In FIG. 3, a panel of 5 mitomycin C-treated 293 transfectants were used to provide secondary stimulation to murine T cells which had been pre-activated with plate-bound parent 2C11 and PVI-1 mAbs for 60 hours and rested for a further 12 hours. The transfectants used were: 1—empty vector (5.0 μg): 2—mem2C11scFv (0.5 μg): 3-mem2C11scFv (0.5 μg)+mem4F10scFv (5.0 μg)+PVIscFv (0.5 μg). T cells were added at 2.5×10⁶ cells per well and 293 cells at 1.5×10⁶ per well. Proliferation was measured by thymidine incorporation between 72 and 84 hours of culture and cytokine levels in culture supernatants after 48 hours. In the upper panels, stimulation with anti-CD3εscFv alone was sufficient to induce significant T cell proliferation and production of IL-2 and IFNγ while cells transfected with empty vector alone induced no response. Co-expression of anti-CTLA-4scFv with an equal amount of anti-CD3εscFv was associated with significantly reduced proliferation and cytokine production. In the lower panels, combined expression of anti-CD3εscFv and anti-CD28scFv resulted in approximately ten-fold enhancement of proliferative response and production of IL-2 and IFNγ compared to anti-CD3εscFv alone as well as detectable production of IL-4. All these responses were significantly reduced by co-expression of anti-CTLA-4scFv on the stimulating cells. Results are expressed as mean±SD of six identical wells for each condition.

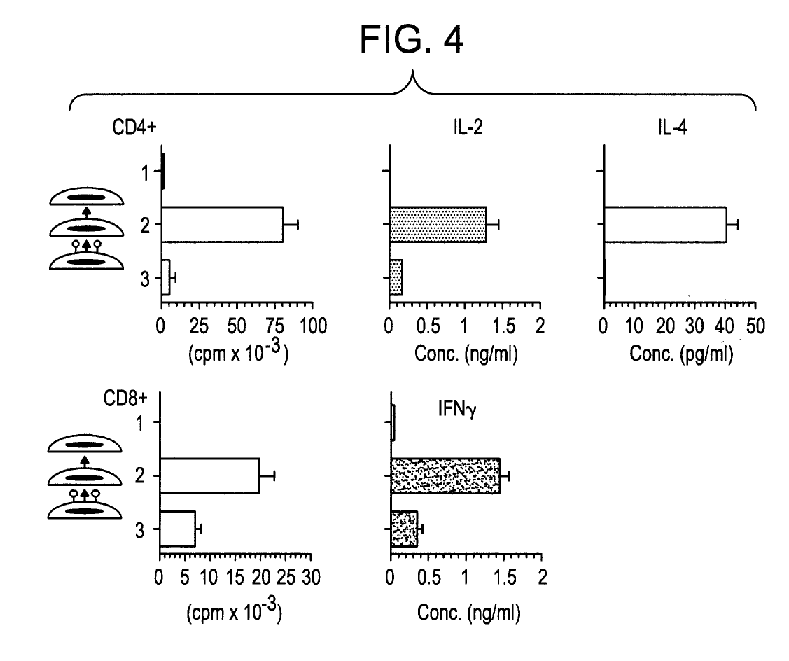

As with primary stimulation, cells transfected with empty vector alone did not induce proliferation or cytokine production. The presence of a TCR ligand was sufficient, however, to induce proliferation and production of IL-2 and IFNγ by these cells. Co-expression of anti CLTA-4scFv was associated with significant reductions in each of these parameters. The addition of low level expression of memPVIscFv resulted in approximately ten-fold increase in the measured responses while the magnitude of the anti-CLTA-4 effect was similar to that seen in the absence of additional CD28 engagement. Expression of anti-CTLA-4scFv was associated with reductions in proliferation and IL-2 production of 80% and 67% respectively with mem2C11 scFv alone and of 66% and 69% with combined mem2C11 and memPVIscFvs. The data shown in FIG. 3 represents one of eight co-culture experiments which were carried out using pre-activated T cells. In all experiments evidence of reduced T cell activation response was observed in the presence of anti-CTLA-4scFv. The range of reduction among experiments was 33 to 84% for proliferation (8 experiments) and 30% to 100% for IL-2 production (4 experiments). Reduction in IL-4 and IFNy production by pre-activated T cells were also observed in two additional experiments. FIG. 4 illustrates the results of a similar experiment in which purified populations of CD4+ and CD8+T cells were pre-activated as before and re-stimulated by mem2C11 scFv alone or in the presence of mem4F10scFv. In FIG. 4, purified CD4⁻ and CD8⁻ T cells were pre-activated with plate-bound parent 2C11 and PV-1 mAbs for 60 hours and rested for a further 12 hours. A panel of three mitomycin C-treated 293 transfectants, plated at 1.5×10⁻ per well, were then used to provide secondary stimulation. Pre-activated T cells were added at 2.5×10⁻ per well. The transfectants used were: 1—empty vector (5.0 μg); 2—mem4F10scFv (5.0 μg)+empty vector (5.0 μg): 3—mem2C11scFv (0.5 μg)+mem4F10scFv (5.0 μg). Vector transfected 293 cells did not induce proliferation in either CD4⁺ or CD8⁺ populations while expression of anti-CD3ε alone was associated with a proliferative response in both subsets and with the secretion of detectable levels of IL-2 and IL-4 (CD4⁺ only) and IFNγ (CD8⁻ only). Co-expression of anti-CD3ε and anti-CTLA-4scFv resulted in marked reduction of proliferation and cytokine production for both T cell subsets. Peak proliferation shown occurred between 64 and 76 hours of co-culture for CD4⁺ T cells and between 30 and 42 hours for CD8⁺ T cells. All cytokine levels were measured following 48 hours of co-culture. Results are expressed as mean±SD of six identical wells for each condition.

TCR and CTLA-4 co-engagement was associated with significant reductions in peak responses of both subsets of T cells. Thus, selective engagement of CTLA-4 upon re-stimulation of activated T cells results in a substantial inhibition of their proliferation and cytokine production. Moreover, during secondary stimulation of bulk T cells and T cell subsets this effect can be mediated both in the presence and absence of CD28 ligation.

### Example 4

### Co-Ligation of CTLA-4 and TCR by Surfaced-Linked scFvs Result in Reduced Tyrosine Phosphorylation of Components of the Proximal TCR Signaling Apparatus

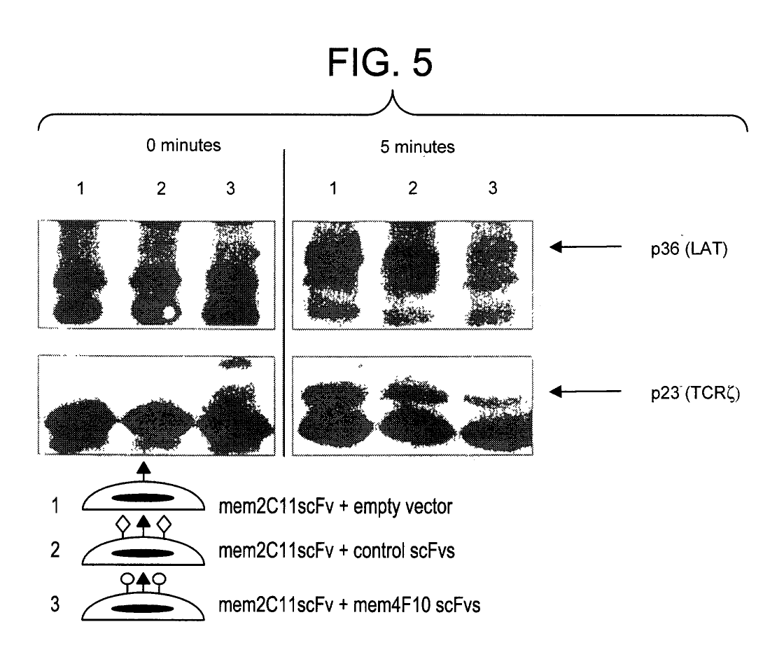

The elucidation of the molecular mechanisms underlying the negative regulatory function of CTLA-4 remains central to understanding its role in normal and abnormal immune responses. A model in which tyrosine-phosphorylation-dependant elements of the proximal TCR signaling complex are modified by recruitment of a CTLA-4 associated phosphatase has been proposed and we have recently reported experimental evidence to support this concept. Using our artificial APC system we next sought to characterize intracellular protein phosphorylation events in T cells receiving an activating signal through the TCR during a cell-cell interaction in the presence or absence of CTLA-4 co-ligation. It was reasoned that pre-activated T cells with substantial intracellular stores of CTLA-4 would be most appropriate for observing the effects of CTLA-4 engagement on early T cell signaling events. Thus pre-activated T cells were incubated in suspension with 293 transfectants expressing mem2CHIIscFv alone or in combination with control or mem-4F10scFvs. Tyrosine phosphorylation patterns were examined after defined periods of stimulation by immunoprecipitation and immunoblotting of cell lysates. The effect of co-expression of control scFv or mem-4F10scFv on these early phosphorylation events is shown in FIG. 5.

In FIG. 5, pre-activated T cells were co-incubated with equal numbers of a panel of three 293 transfectants at 37° C. The transfectants were: 1—mem2C11scFv+empty vector (5.0 μg): 2—mem2C11scFv (0.5 μg)+control scFv (5.0 μg): 3—mem2C11scFv (0.5 μg)+mem4F10scFv (5.0 μg). Following anti-phosphotyrosine immunoprecipitation and immunoblotting of whole cell lysates two prominent protein bands of approximate molecular weight 36 and 23 kD were induced with peak induction following 5 minutes of co-incubation. As shown, the intensity of these bands, which correspond to LAT (p36) and TCR (p23), was mildly reduced at 5 minutes in the presence of controlscFv when compared with empty vector. In contrast the presence of mem4F10scFv was associated with a substantial reduction in tyrosine phosphorylation of the two proteins at 5 minutes. Photodensitometric analysis of the protein bands induced in the presence of control scFv compared with that of anti-CTLA-4scFv demonstrated 35% reduction in intensity for LAT and 85% reduction for p23 TCR when CTLA-4 was engaged.

Induction of tyrosine phosphorylation of specific proteins was induced following co-incubation of the T cells with antiCD3_{ε-}expressing cells with maximal intensity after five minutes. The most prominent of these correspond in approximate molecular weight to two early components of the TCR signaling complex (hyperphosphorylated (p23) TCR) and longer for activated T cells (p26. LAT). Induction of tyrosine phosphorylation of these proteins does not occur on incubation of pre-activated T cells with untransfected 293 cells and in separate experiments immunoblotting specific for TCR and LAT has confirmed their identity (data not shown). Significantly, while co-expression of control scFv is associated with levels of TCR and LAT phosphorylation which are comparable to those with mem2C11 scFv alone, co-expression of mem4F10scFv results in a substantial reduction in tyrosine phosphorylation of both proteins. This observation correlates with the attenuation of subsequent proliferation and cytokines production seen in co-culture experiments and is in keeping with our reported findings that a physical association between CTLA-4 and elements of the proximal TCR apparatus mediates their dephosphorylation.

### Example 5

### CTLA-4 and TCR Ligation Must Occur on the Same Cell Surface for Optimal Negative Regulation to occur, Whereas CD28 can Function In cis or In trans

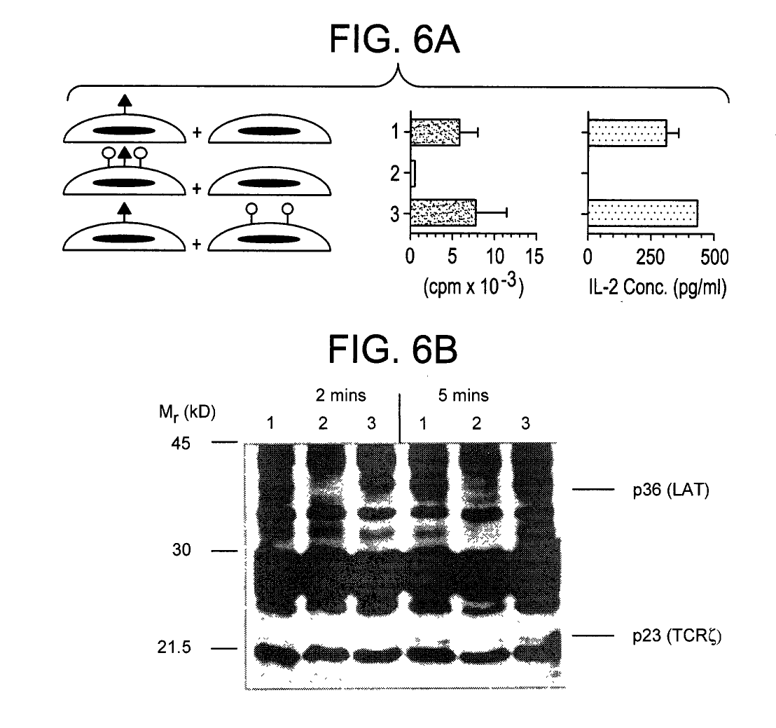

One prediction of a model in which the negative regulatory effect if CTLA-4 during a productive T cell/APC interaction is exerted upon proximal TCR signaling is that engagement of CTLA-4 at a point distant to that of TCR engagement would fail to attenuate activation events. In contrast, a model in which negative effects are mediated wholly or partly through an independent signaling pathway would predict the separate ligation of CTLA-4 would result in a qualitatively similar outcome. In order to address this question we compared the effect of mem4F10scFv expression on the same cell surface (“in cis”) as the antiCD3ε ligand with that of expression on a separate, admixed, population of cells (“in trans”). Once again pre-activated T cells were employed and a secondary stimulation was provided through the TCR alone. FIG. 6, Panel A shows the results for proliferation and IL-2 secretion.

Pre-activated T cells were co-incubated with the mitomycin C-treated 293 transfectant mixtures in order to compare the effects of expressing mem4F10scFv on the same cell as mem2C11scFv or on adjacent cells. The mixture used were: 1—mem2C11scFv (0.5 μg)+empty vector mixed with empty vector 4 μg): 2—mem2C11scFv (0.5 μg)+mem4F10scFv 5 μg) mixed with empty vector 4 μg: 3—mem2C11scFv (0.5 μg)+empty vector 5 μg) mixed with mem4F10scFv (5 μg). In panel A, 293 transfectants mixed with 1:1 ratio were added at a total of 2×10⁴ cells per well and pre-activated T cells at 2.5×10⁴ per well. Proliferation between 60 and 72 hours and IL-2 concentration at 48 hours are shown. When the CTLA-4 ligands was expressed on the same cell surface as the TCR ligand a significant reduction in proliferation was seen and IL-2 was undetectable while expression of anti-CTLA on a separate population of cells was associated with mild enhancement of both proliferation and IL-2 concentration. Results for thymidine incorporation are expressed as mean±SD of six identical wells for each condition. In panel B, anti-phosphotyrosine immunoblot following phosphotyrosine immunoprecipitation of three sets of whole cell lysates. Pre-activated T cells were incubated in suspension at 37° C. for 5 minutes with three sets of 293 transfectant mixtures at 1L1 ratio. Strong induction of tyrosine phosphorylation of protein bands corresponding to LAT (p36) and hyperphosphorylated TCR by mixtures 1 and 2 was seen while mixture 3, in which TCR and CTLA-4 ligands were co-expressed on the same cell population induced markedly less phosphorylation of these proteins.

When TCR and CTLA-4 engagement are provided on the same cell surface a significant attenuation of proliferation and IL-2 production is observed. When the two ligands are provided in trans, however no negative effects occur and a modest enhancement of proliferation and IL-2 secretion is seen. By mixing pre-activated T cells with similar combinations of transfectants in suspension and carrying our phosphotyrosine immunoprecipitation and Immunoblotting on cell lysates we were also able to demonstrate the absence of an effect of CTLA-4 engagement in trans on early TCR signaling (FIG. 6, Panel B). These results show that the interaction of CTLA-4 with cell surface ligands may result in widely differing outcomes depending on the point of ligation relative to that of the TCR. Once again this implies that a physical association between CTLA-4 and the TCR underlies the primary mechanism of negative regulation.

### Example 6

### Surface-Linked Anti-CTLA-4scFv Attenuates Proliferation of CD4+ T Cells When Co-Expressed with MHC/peptide Complex

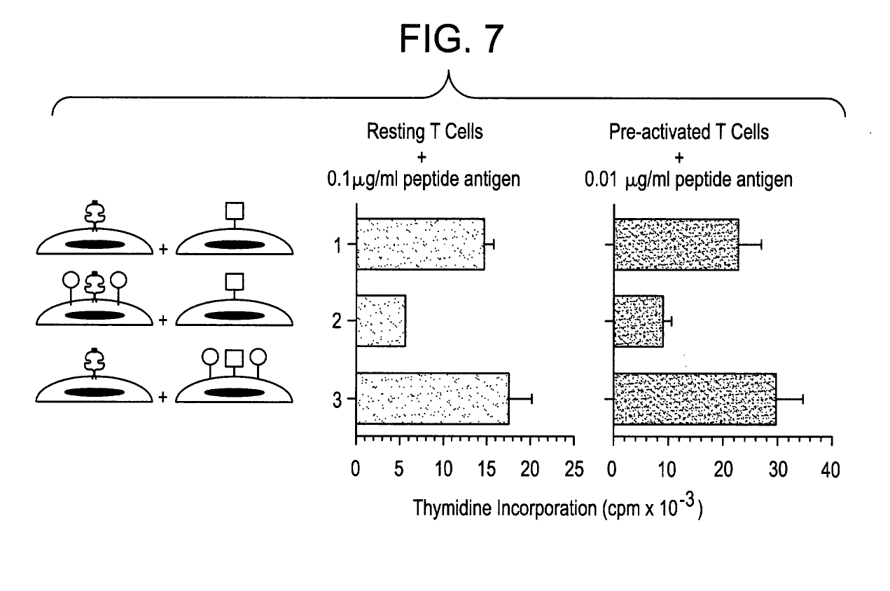

Although multiple studies have been published demonstrating the ability of CTLA-4 ligation to attenuate antigen non-specific (largely anti-CD3ε mediated) T cell stimulation, limited data is available to support similar effects on bonafide antigen-specific responses. Therefore, a modification of the system was used to examine the effects of the membrane-bound anti-CTLA-4 scFv on primary and secondary activation in a well-characterized model of antigen-driven T cell activation. T cells derived from mice transgenic for the DO11.10 TCR (with specificity for the ovalbumin-derived peptide OVA ₃₂₃₋₃₃₉ presented by the murine ClasslI MHC I-A^{d}) were co-incubated with 293 cells transfected with cDNAs encoding the α and β chains of I-A^{d} and pulsed with antigenic peptide. A second population of 293 cells transfected with memPVIscFv were a mixed to provide CD28 mediated co-stimulation “in trans”. In this way the level of the TCR signal could be accurately fixed by addition of exogenous antigen. Furthermore, in providing the CD28-legand on a discrete population of cells it was possible to separately observe the effects of CTLA-4 co-ligation with either the TCR or with CD28 by co-expression of mem4F10scFv on one or other cell surface. FIG. 7 shows the results of a proliferation assay using both resting and pre-activated DO11.10 T cells.

In FIG. 7 resting and pre-activated lymph node T cells purified from DO11.10 transgenic mice were incubated with three different combinations of 293 transfectants mixed at 1:1 ratio as shown, in the presence of OVA_{323 339} (0.1 μg/ml for resting and 0.01 μg/ml for pre-activated T cells). For each mixture one transfectant expressed 1-A^{j} (10 μg each of the α and β chain cDNAs) along with empty vector (5 μg) or mem4F10scFv (5 μg) and the other expressed memPVIscFv (2%g) also with empty vector or mem4F10scFv (5 μg). Resting T cells were added at 10³ per well, pre-activate T cells and between 60 and 72 hours for pre-activated. In both experiments incubation of DO11.10 T cells with cells separately expressing significantly reduced by co-expression of anti-CTLA-2scFv on the same cell surface as the MHC/peptide complex (2). Co-expression of anti-CTLA-4scFv on the same cell surface as antiCD28 (3) resulted in a mild increase in proliferation. Results are expressed as mean±SD of six identical wells for each condition.

Proliferation of both populations of DO11.10 T cells was induced by a mixture of peptide-pulsed I-A^{d-} cells and memPVIscFv-expressing cells (incubation of purified DO11.10 T cells with 293v cells not expressing I-A^{d} does not induce proliferation even in the presence of a high concentration (1.0 μg/ml) of peptide as well as the CD28 ligand). As with anti-CD3ε-mediated TCR stimulation co-expression of mem4F10scFv with the MHC/peptide complex was associated with significantly reduced proliferation of resting and pre-activated T cells. In contrast when CTLA-4 was engaged on the same cell surface as CD28 the result was a modest increase in both experiments. The magnitude of the inhibitory effect of CTLA-4 engagement in cis with TCR/MHC interaction was most prominent at submaximal concentrations of people antigen being proportionately less in the presence of higher antigen dose for both primary and secondary stimulations. These result demonstrate the ability of CTLA-4 engagement by surface-linked scFv to negatively regulate the antigen-specific activation of CD4+ T cells when co-expressed with peptide/MHC complex. In addition, when TCR and CD28 signals are engaged on separate cell surfaces the regulatory function of CTLA-4 can be seen to “co-localize” with the TCR suggesting a lack of effect on TCR-independent elements of CD28 intracellular signaling.

The practice of the present invention employs conventional techniques of molecular biology, microbiology, recombinant DNA and immunology, within the skill of these arts. Such techniques are found in the scientific literature. (See, e.g., Brock, Biology of Microorganisms, Eighth Ed., (1997), (Madigan et al., eds.), Prentice Hall, Upper Saddle River, N.J.; Sambrook et al., Molecular Cloning: A Laboratory Manual, Second Ed., (1989); Oligonucleotide Synthesis, M. J. Gait Ed., 1984, Animal Cell Culture, Freshney, ed., 1987; Methods In Enzymology, series, Academic Press, Inc.; Gene Transfer Vectors for Mammalian Cells, Miller and Calos, Eds., 1987; Handbook of Experimental Immunology, Weir and Blackwell, Eds., Current Protocols in Molecular Biology, Ausubel et al., Eds., 1987, and Current Protocols in Immunology, Coligan et al., Eds., 1991)). These references are incorporated in their entirety herein by reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A construct for downmodulating an immune response in a subject, said construct comprising an exposed surface, wherein said exposed surface has attached to it i) an antigen-binding portion of an antibody that binds to a CTLA-4 molecule that is expressed on a T cell of the subject, and ii) an MHC molecule selected from the group consisting of: a class II molecule that is syngeneic to the subject, a class I molecule that is syngeneic to the subject, and a class I molecule that is allogeneic to the subject.

2. The construct of claim 1, wherein the antigen-binding portion is a single chain Fv (scFv) molecule.

3. The construct of claim 1, wherein the single chain Fv (scFv) molecule binds to human CTLA-4.

4. The construct of claim 2, wherein the scFv molecule is humanized.

5. The construct of claim 1, wherein the antigen binding cleft of the MHC molecule comprises a peptide for which the immune response is specific.

6. The construct of claim 1, wherein the construct comprises a lipid bilayer.

7. The construct of claim 6, wherein the construct is an acellular construct.

8. The construct of claim 6, wherein the construct is a cell.

9. The construct of claim 8, wherein the cell is a eukaryotic cell.

10. The cell of claim 9, wherein the cell is syngeneic to the subject.

11. The cell of claim 9, wherein the cell is allogeneic to the subject.

12. The construct of claim 1, wherein the antigen-binding portion of an antibody that binds to a CTLA-4 molecule is attached to the exposed surface via a phosphatidylinositol-glycan anchor.

13. The construct of claim 1, wherein the antigen-binding portion of an antibody that binds to a CTLA-4 molecule is attached to the exposed surface via a transmembrane domain.

14. The construct of claim 1, wherein the antigen-binding portion of an antibody that binds to a CTLA-4 molecule is attached to the exposed surface via a chemical linkage.

15. The construct of claim 1, wherein the construct does not bind to CD28.

16. A method of downmodulating a primary immune response in a subject comprising administering the construct of claim 1 to the subject such that an immune response in the subject is downmodulated.

17. A method of downmodulating an ongoing immune response in a subject comprising administering the construct of claim 1 to the subject such that an immune response in the subject is downmodulated.

18. A method of downmodulating a immune response in a subject comprising causing a cell of the subject to express an antigen-binding portion of an antibody that binds a CTLA-4 molecule, the CTLA-4 molecule that is expressed on a T cell of the subject, such that the immune response in the subject is downmodulated.

19. The method of claim 18, wherein the antigen-binding portion is a single chain Fv (scFv) molecule.

20. The method of claim 19, wherein the wherein the single chain Fv (scFv) molecule binds to human CTLA-4.

21. The method of claim 19, wherein the the scFv molecule is humanized.

22. The method of any one of claims 16-18, wherein the immune response is against an self antigen.

23. The method of any one of claims 16-18, wherein the immune response is against an non-self antigen.

24. The method of any one of claims 16-17, wherein the immune response is against an allogeneic antigen.

25. The method of any one of claims 16-18, wherein the immune response is mediated by CD4+ T cells.

26. The method of any one of claims 16-18, wherein the immune response is mediated by CD8+ T cells.

27. The method of claim 18, wherein the cell is a professional antigen presenting cell.

28. The method of claim 18, wherein the cell is further caused to express an MHC class I or an MHC class II molecule.

29. The method of claim 18, wherein the cell is transfected with a nucleic acid molecule encoding the antigen-binding portion of an antibody that binds CTLA-4.

30. The method of claim 29, wherein the cell is transfected ex vivo.

31. The method of claim 29, wherein the cell is transfected in vivo.

32. A method of preparing an allogeinc cell for transplantation into a subject comprising causing the allogeneic cell to express an antigen-binding portion of an antibody that binds a CTLA-4 molecule expressed on a T cell of the subject to thereby prepare an allogeneic cell for transplantation into a subject.

33. A method of transplanting an engineered allogeinc cell to a subject comprising: causing an allogeneic cell to express an antigen-binding portion of an antibody that binds a CTLA-4 molecule on a T cell of the subject to create an engineered allogeneic cell, and administering the engineered allogeneic cell to the subject such that the engineered allogeneic cell is transplanted to the subject.


SEQUENCE LISTING (4 sequences)

SEQ ID NO: 1 - DNA, 672 bp, Homo sapiens
atggcttgccttggatttcagcggcacaaggctcagctgaacctggctgccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgacggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 2 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGAQIYVIDPEPCPDSDFLLWILAAVSSGLFFYSFLLTAVSLSKMLKKRSPLTTGVYVKMPPTEPECEKQFQPYFIPIN

SEQ ID NO: 3 - DNA, 3806 bp, Homo sapiens
taaagtcatcaaaacaacgttatatcctgtgtgaaatgctgcagtcaggatgccttgtggtttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaggaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatggcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaaatggagataatggttacaaatgtctcttcctatagtataatctccataagggcatggcccaagtctgtctttgactctgcctatccctgacatttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccagatcgaaaatactgtactttggttgatttttaagtgggcttccattccatggatttaatcagtcccaagaagatcaaactcagcagtacttgggtgctgaagaactgttggatttaccctggcacgtgtgccacttgccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactact

SEQ ID NO: 4 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS